SEQUENCE LISTING (3 sequences)

SEQ ID NO: 1 - protein, 744 aa, Chlorella vulgaris
  MISC_FEATURE (1)..(744): Mutation in an ABC transporter (Arg347Cys) of a modified strain
MWSAGLQFQASATKLPSRRRPLPAARPPCLVAKRHAAAGAVRQQQQRWPWRTIPRAAALAAAAAAAADDPSATQPALIDGDDAAAAATGGVAASTALAPTPAAAADVGLSLSELWQLLQQDRRRLAACIACTAVSVASAVLVAPCLGRVVDIISRGTAATPRELAVAVGRLGTVYVIANVGMAVQVALSLALGEGLAHRLRCRLFGALLSRDTLFFDQVKTGQMVAWLGQDVEVLQSTVSKLLGARGIRSAFETCGIIIVLFTLSWPLAAALLVSAPLLTPLIARLSSAIGIASKASQAASSEVSAAADEVVENMRVVKLFAQQQAELKRFQGLLGTAHQLALKVLCLQALLDGSSRVRNTLCVLATLGLGAYMALHSAVSIGTCYSFFVFSFSFAFALGNLTNTVGDVARAAGAINRAMRTMQQALGTASTEATAALLSSADNAPLEAGGDAVTGAQHGRQLPAGWRGEIEFQNVGFSHPGGWAIKDLSFKLPPGSTVALVGPSGGGKTTIASMLMKLYDVHAGHILVDGVPLDQLDTKWWRQQLGVVMQSPGLLTGRIADIIRYGQPGASDADAARAARAAQADGFIEALPNGYQTVIGSGSGIELSGGQQQRLAIARALLPRPRLLIFDEATSALDVETEQGVTQALEGAGRGVTSLVIAHRLSTVRRADLIVVVASGKVVEQGTHEQLMQRQNGVYWSLVSGAESRGQDSTEDDDEDDEEQDVQGEGKQPPMAHELAPTA

SEQ ID NO: 2 - DNA, 5313 bp, Chlorella vulgaris
  source (1)..(5313): Mutation localised at a splicing donor site after exon 9 at the end of the WD40 repeat domain at a position 2750 of a gene encoding for a guanine nucleotide-binding protein in modified strain
atggcctccgctggcggcgcggtgccgccacgcaaagtgtcgttcctctttcggcgcgagggcataggggtgggtacttgcaggagtgcagaggttgtgcttggcggtggcttacagtcaaacgcagaaggacagcgtcgctcatctggttgccccggtccccatgcctcccccttgccgccgcatcaatgttcctgcaccctcccctcccatcctcacgcagcactgcggaggggtgaatgccctggctttatccagggacgaacagcagctgtggaccggttccagggactcaaccatctgctggtgcgaggcagctgcgctggccagcagtacgctgcgcttggtcgcaaccctcctttgccagtcgggtgccagcagcattttcgcctctcagcttcaagctacagcgtcatgttctcagtcacgtcatgtgtcctgcctgcagctggtccacagctgagaaagcgcagccggatctgatgcacgagcacgtcggtcacattgactgggtgggtgttgcaggcgccctcgtcacaccctcgtgtgcttgtgcatgtatgctcaaatgtcgcgttggatcatcacagcgttgtgtggttgtaaggggtctcatgggcacctgcactgctcttcattggagctcagcagggcttcatgctgcgtggtgttgatgccagctctctccaagcagccccagcaaaccagaagatgcaccaccctctgccccagtgctgacctccagcctgctgtgccgtgatgtgtccgcaggtcaatgggctggcactggcaggcgatgtgctggtgtcctgctcctccgacaaaacgctgcgggtgtggcagcccgactcgccaggtcagcagcacgtgccagctcagctttggctgtgctggccgcctctgcaactgctgcaggtgtttgctctccgcttttgccaaaggagtctcgcctgccccaccctggcccaacctagtcgctcacccaccccacccgcttcacactttgtgtccccccccagcagagccagcgctgggctgcctgcagggacacagcgactatgtcaccgcgctggctgcgtgcgccgacggcggcgtgctggcctcgggcggcctgcgtggcgaggtgctgctgtgggacctggccacgctgcggggcatcatggcgggcagcacccaggtgaggctttgagtgctgccagctgtcctgtgctgccgacctgcagctgctgcccacctgcagctgctacatgcaccggctgcactgatatggctctcttgattacagacaatgcacaatcatacacctgtaagacaattgtggtcataaatcatgtgcgcaggaccaaggttacagcgcatcaacagcggaggtgccccgtgcttcagtgtatgctgtggccatcaatgctgctggcacgctgattgcggcagggtcgacccaggtgcggcacgacctgggggctggacagtgcagcagtgtcgaatggcagctgcagagctgaaggatgcagactcggcttgcaggctgcacgtgtgacgattctcacggccgacactgctgtctgttgcgatgtttccgatgtgctcctgcctgcacattcgacctttgtacttctccggcttgcttgatgcgatgtgctgtgcagggcgtgagcgtggtggacgtccgcagcgggcgcagcgtgatgcagctcaagggccacacagacaacgtcaggtgtgtgtgtgtgggggggggggggggtggcggtggggagtgtgtggcgcagtgtgtgcgttggagtgttctgttgctgcttcctttccaaagcgcttgcagctctgggtgatggctgccccttctgtggtctcagcacaccctttcctcacacaggtgccgtgcgcctggtgttgcctggtgcacacctgttgtttttcccgaaaccttggggtgccattccaattccgagcgtgctgcaccgctcaacctcaccccaccaccacccagcaccgcattggtgccgactgctctaatcgtggttgttgttgaatcactgcctgtctgcaggtcgctgcagcttgaagcagaggggcggctgcttctgagcgggtctgcggaccatacaatgcggctgtgggacctgggccagcagcgctgtgtgcagacgctggccgtgcacactgacagcgtgtgggcgctggcggccaccccctcctttgcggcggtgttcagcggcgggcgggacggctgcatataccggtgggtgcattcagatgtgtgtgtgtgtgtgtctgtggtggtggtggggagtgtggtggtgcggccggcggctgtgccagtggggacaccctgtgacggcatgaccttgcgggttccctctctgcctcttgcccaccatggctgctgacccatcatgtcactgcctccctgtcactccaccaccctgcagcacccgcctgtcgacccgggttagcgagctgctggctgtggagcagcggccggtgctggcgctggctgtggcctcggacgagcggcggctgtgggcagccaccactgcgcccagcgtgcacacctggcacctggagttggctgcagcagcttcggcacacaccgcggcgataggcggcggcagcaccagcgttggcggcggcacgccgcggtcgccgctgggggctgccgccggggctgccgccgtgctgggcggtccctgcttcattgccagcccctctgcagccgcccgagcacgactaccgtttgctggacagcaggatgaggcactgttgctgctgactgcgctgcctgctgctgctatgctgctctgattgcaaatggtgccgccgctggtccagcacctgtctcccatcacgccttccagtgtcctgtttgattcgccgccattgtaatgcaacagaccgccagccatcgtccttgctgcctgccggcctgcaggctcccatccaccgccgcagcagggcgagccagccgccgccacgcccggtctgccgccaattcagcatgccgtggtgctcactgacaggcggcatgtgctgacccgagatgcggatggcacagtgctgtgctgggacctcacagcaggtgagatggggtgtgcagtgcttgggcggcaggcactcacacagctgacagaagttgctggatacactgtcgcacgtgcagcctacgtcgctttatcaaacaatctgcattgtatccaaccagacaatagaatctgcaaccgtatcccaccccacacgccccgccctcactcaccctgcaggctgtgtgacccgcgactatggtcgctgcgagttgaaagaggtggaggcggcactcttccagcccaaccactctgtatctgcctggtgccagcccgaggtcaagctgggcagcctggggggtgccatggagccaccgcagtgctttggtgctgagatctatgcccaggtgcggcagttgtttccagatactcatgtcacactgtttcaagcttaagcgctgcaggctggtgcacaaattccactcattacacgacttctgttcaggatgccgccccatgtttcttttgctccagatatcactttgcgtgtgaatgagcaagtctggcgttgccatgcctagctcgccctgctcctgctttctcctcgtgtcatgccacctccatccgcgtcgttgcacctgcaggacctgggagacgaggaggcacctgtcgacttgaaagtcaactttggggagcagatgctggtggctttattcaagggctgggcagaagcggcgcagcgcagcagctgccacgctctgcctgctccctccacgcaagttctgcggccagcacaagagcgggtcctgggcagccgggcagtagaccagcagtctggtgctgagccaagcgacagcggcggcagccacggcggcgatggcgagcggcaaagcaatgtggcggcggagcaggcggcagcgggggggtgcgaacgcagcaccctgcaggatggtgacagtgaggggcagagtattgacgggtgagcggcagcatgcaccgcacttgcgcagctatagtcagcgatggatggaagcatagtcatgacagagctgtgggtgtaggcacctgcaagatggaggctttctttctgaatttcgggccggcggtgttcaggcttggccggccatcctgctcttgtcctggcagcaagtactcgcaaacctgcacgtgtgtgtgagtgctgcggcgtcctgctcgtgcactggtcgctcaccctgccagcctgccctcctcgccacacagggagcccgaccaagccactcgcttcaagtttctcaccccgctggcacctgtggtgatggtcagcggcagtggcggcagcggtaccagagctggcggtaccgtgccctggaggtgccctactgatgcgttcaccgggaaggaagacgtgcctcaggtgctgtccggggtcgtgtcaggaaggaagctgcggctgggggcccgacagcaccagaggcaaccagagcacttggcatttgcacgcctttcgacgctctggtgtagctgttgactctggcccgcttgctgcctcctgccctgctctgccgcagtgggtagccgactgcgtcctacggggccggtatccggtcagcagggagctcaagatggcgttccagctgatgcccctgccggtaagcccttgcatacaagtagtggggctgataactgccagcttgcacgatccagcagctgagaacagtgaagcgctttgcagcgcaaatgctactgtggcgcagtgccaagccgtcgtgactcatctcaaacccctctctcaaccgccctcactatacaccgtttgcctgcctgcagggctcgccgctgccatcgctgctgcaaagcaagctgaatgcaccgagggtgctgcgggtcaacaaggtggctgactacatccgccgcaagatggaggagcagggcatagcattgcgggaggagccgctgttttgggatggcagcaaggaggagaagtggcacgcggagcaagctgccagtgccgacggcagcaacgacggcagcagtgggggccttgtctcactgccggtgcaccagctgcaccctgtgcagccgcttccgggcgccgccagcagcatcagccccgcggagagggcccggggcagcggtttggccgggcacggagcgcttttggtcacgtgtgcgggcaacactgtgccttgggactttagcttggcagcagtacgccagtggatttggcggcgctctgacgatttggtgctgcattactctgtgagggatcgctccacgccgctgaagctgccagccattcgacgtccttcatgaa

SEQ ID NO: 3 - protein, 471 aa, Chlorella vulgaris
  MISC_FEATURE (1)..(471): Mutation in an aquaporin (Ala46Val) of a modified strain
MSGGTAVNNTYSLAAECGSEMIGTCFVIYLGESIIANELLSKTKGVRMGWLAVGFGLSFGVVIMMLGYISAHLNPATCLALWVIGKIPFTHFLALAGAEFAGAFVGACLVFLHYIPHFKTVPEPMPRNEDDVLLRSRDALTFEALNIASYDTRARQRTASSKGGVMGVINDAIKDVRYYFKETHQAPQEHVELVEVALGPAEMKGAVENRLRRRSVQVCDVHRRLKDMSIEEFKEKLQVKSSALNGGMPRSASINLGELDRAMGGEQGAAEGKDLGDSSSGEEAHQQRVQRQATAPPQVQGPPTAWKDKLMRHIERYGERQARVFDAAVIADQNAKLSIFCTRPAIYAPVFNFLTEVMCTTALVFGALMMYARRDLLNPEHKALFQSYEGMWIGFFVFVAILGLGGPTGIAANPARDFSPRLAHALLPIAGKGPSEWHYGWIPFWAPFFGGAAAGGLYLLVQMLNHSKYVA

(19) United States
(12) Patent Application Publication
(10) Pub. No.: US 2024/0287441 A1
(43) Pub. Date: Aug. 29, 2024

(54) MODIFIED STRAINS OF CHLORELLA MICROALGAE SPECIES HAVING REDUUCED CHITIN CONTENT

(71) Applicant: ALGENUITY HOLDINGS LTD, Stewartby, Bedfordshire (GB)

(72) Inventors: Andrew Pearce SPICER, Stewartby, Bedfordshire (GB); Alexander Findlay PUDNEY, Stewartby, Bedfordshire (GB); Richard Thomas SMITH, Stewartby, Bedfordshire (GB); Gino SCHIANO DIVISCONTE, Stewartby, Bedfordshire (GB); Jean-Charles Eric Francois ISNER, Stewartby, Bedfordshire (GB)

(73) Assignee: ALGENUITY HOLDINGS LTD, Stewartby, Bedfordshire (GB)

(21) Appl. No.: 17/999,961

(22) PCT Filed: May 27, 2021
(86) PCT No.: PCT/IB2021/054639

§ 371 Date: Nov. 28, 2022

(30) Foreign Application Priority Data
May 27, 2020 (GB) 2007940.6
Jan. 14, 2021 (GB) 2100462.7

(51) Int. Cl. C12N 1/12; C12N 15/01; C12R 1/89

(52) U.S. Cl. CPC C12N 1/125; C12N 15/01; C12R 2001/89

(57) ABSTRACT

Disclosed are modified strains of Chlorella microalgae species, the modified strains having a reduced chitin content. Also disclosed is a method for producing them. The method involves performing mutagenesis of a parent strain of the microalgae. Furthermore, disclosed is a composition comprising algae biomass derived from the modified strains and their use in food and/or cosmetics amongst other applications. Advantages of the low chitin content include aiding digestibility and nutrient availability of the whole-cell ingredient without further downstream processing.

### INCORPORATION BY REFERENCE

The sequence listing in XML format is incorporated herein by reference in its entirety.
a. Name of File: ALGEN004US_Sequence Listing_ST25
b. Date of Creation: 27 Mar. 2024
c. Size of File: 18,351 Bytes

### TECHNICAL FIELD

The present disclosure relates generally to algae or microalgae and more specifically to modified strains of Chlorella microalgae species having a reduced chitin content. The present disclosure also relates to methods of producing the modified strains, compositions comprising algae biomass derived from such strains and to their use as food ingredients amongst other applications.

### BACKGROUND

The ever-increasing consumer awareness regarding the environmental and personal health consequences of intensive farming of animal protein and a diet rich in meat is driving the growing market for vegetarian and/or vegan food sources. Plant-based food products, including meat analogues, appeal to a wide demographic of consumers, including those who do not necessarily identify as vegetarian or vegan. In addition to plant-based food products, algae have been identified as potential sources of vegetarian and/or vegan foods.

Algae are simple, non-flowering plants requiring only water, sunlight and a few nutrients for growth thereof. Algae may range from microscopic algae (or “microalgae”, such as phytoplankton) to multicellular algae (or “macroalgae”, such as seaweed). Macroalgae, such as seaweed and kelp have been traditionally used as a food source for both human and animal consumption. Beneficially, innovative product formulations have been successful in delivering algal biomass as whole food or food ingredients.

Recent trends in nutraceuticals and food industries have identified microalgae as a potential source of essential nutrients that provide several other benefits. The green microalgae, Chlorella vulgaris, has been produced commercially as a food and dietary supplement for at least the last 50 years. Historic export of Chlorella products from producers in southeast Asia, to the European market and consumption as a food ingredient or supplement is the basis of Chlorella vulgaris exemption from EU Novel Food Regulation (EU) 2015/2283—being as it was “on the market as a food or food ingredient and consumed to a significant degree (within the EU) before 15 May 1997”. In addition to being safe to eat for both humans and animals both as a whole food and as an ingredient, Chlorella vulgaris is also present on the CIRS China List of approved cosmetic ingredients both as whole cell and as extract, as well as being included on the European Cosmetics Ingredients list. Besides Chlorella vulgaris, other species of Chlorellae such as Chlorella sorokiniana, as well as microalgae related to Chlorella genus especially those selected from the family Chlorellaceae, may be exploited commercially for various applications for example in food, nutraceuticals, cosmetics, and so on.

Despite the nutritional and other advantages offered by this ingredient, Chlorella vulgaris naturally possesses a tough, poorly digestible cell wall. Specifically, the cell wall of Chlorella vulgaris is comprised of a hemicellulose component and a “rigid” component, which is mainly composed of glucosamine in a chitin-like polysaccharide (Baudelet et al. 2017; DOI: 10.1016/j.algal.2017.04.008). The “rigid wall” fraction constitutes between 60 and 66% of the overall cell wall (Abo-Shady et al. 1993; DOI: 10.1007/BF02928041).

The glucosamine component of the cell wall of Chlorella vulgaris is essentially chitin (a homopolymer of β-(1,4)-N-acetyl-D-glucosamine) or a chitin-like polysaccharide (such as chitosan, produced by diacetylation of chitin), synthesised by a chitin synthase enzyme. Typically, chitin is a structural polysaccharide commonly found in the cell wall of species such as shellfish, insects, fungi, yeast, worms, mushrooms, and marine invertebrates. Interestingly, chitin is not a typical component of the cell walls in all green algae and the presence of chitin in Chlorella species might be due to horizontal gene transfer from Chlorella-infecting viruses (Blanc et al., 2010; DOI: 10.1105/tpc.110.076406).

Generally, the rigidity of the cell wall of Chlorella vulgaris due to its chitin content, impacts digestibility and nutrient availability for human or animal consumption. Some of the animal population and the majority of the human population are able to digest chitin due to the presence of genes for “acidic” chitinase or CHIA (Janiak et al., 2018; DOI: 10.1093/molbev/msx312), in addition to the action of their gut microflora. Interestingly, eating more chitin reportedly increases expression of chitinase in a range of livestock and domestic animals that have functional CHIA genes (Tabata et al., 2018; DOI: 10.1038/s41598-018-19940-8). However, only 80% of human subjects in one study (20 out of 25 subjects) had assayable CHIA activity in their gastric juice, and furthermore, this activity varied within this group by 172-fold (Paoletti et al., 2007; DOI: 10.1159/000104144). This is indicative of a variable ability to digest the chitin component of Chlorella vulgaris biomass by humans before it reaches the gut and explains why this product is typically “cracked” before sale.

In order to overcome the problems associated with the rigid cell wall of the Chlorella vulgaris and to promote their use as whole foods or food ingredients by improving its digestibility and bioavailability, Chlorella vulgaris biomass may be processed to break the cell wall. Conventionally, such processing of Chlorella vulgaris biomass includes pulverizing, milling, breaking, grinding, cracking or extruding. Existing Chlorella products for human consumption are frequently sold as being “cracked” or “pulverized”, a physical downstream process following cultivation, before packaging. The downstream processing of Chlorella vulgaris biomass physically disrupts (i.e., by mechanical means) the tough and poorly digestible cell wall of Chlorella vulgaris.

These downstream processing techniques increase the overall cost of production, being energy-intensive while also negatively impacting on sustainability. Furthermore, the downstream processing techniques do not address the overall content of chitin or chitin-like polysaccharide in the Chlorella vulgaris biomass.

Another disadvantage associated with the rigidity of the cell wall of Chlorella vulgaris is its negative effect on the genetic transformation efficiency of the strain. In this example, the rigid cell wall acts as a physical barrier to recombinant DNA, which directly impacts on the ability to undertake advance genetic engineering of Chlorella vulgaris for biotechnology applications.

It is the object of the invention to provide a non-genetically modified Chlorella vulgaris strain with improved digestibility, for use in consumer products.

### SUMMARY

The present invention overcomes the highlighted drawbacks by using a whole algal cell having a reduced chitin content. The algae strain of the invention is produced as the result of mutagenesis affecting existing metabolic pathways, rather than by a recombinant method. The invention is therefore a non-genetically modified whole algal cell having genetic stability in producing a cell wall with reduced chitin content. In particular, the modified strains of Chlorella vulgaris and other Chlorella microalgae species having reduced chitin content enable improved digestibility to consumers as well as resulting in improved genetic transformation efficiency of the strains. The invention also relates to whole-cell algal ingredients and their applications. The reduction of chitin content in the Chlorella vulgaris strains reduces the need for additional downstream processing of the Chlorella vulgaris biomass, therefore, lowering the energy intensity of the overall manufacturing process.

One aspect of the invention is a modified strain of a Chlorella microalgae species, derived from a parent strain of a Chlorella microalgae species, the modified strain having a chitin content at least 10% lower than the chitin content of the parent strain of Chlorella microalgae from which it is derived, when grown under the same conditions. The modified strain of Chlorella microalgae species is obtained from the parent strain, by performing mutagenesis.

One aspect of the invention is a modified strain of Chlorella vulgaris having a chitin content of less than 4.8 mg/g dry cell weight, preferably in a range of 0.001 to 4.8 mg/g dry cell weight. The reduction in chitin content results in higher overall digestibility, which is beneficial when the algae are used as food ingredients. We have shown that the strains of the invention have a protein digestibility-corrected amino acid score (PDCAAS) in a range 0.75 to 1 (75% to 100%) (as calculated using the standard Dumas method for protein assay and a nitrogen to protein conversion factor of 6.25). Preferably the PDCAAS is in a range of 0.75 to 0.831 and most preferably in a range of 0.75 to 0.79.

Typically, the modified strain of Chlorella vulgaris has a greater than 10% reduction in chitin content, compared to the parent strain of Chlorella vulgaris grown under same conditions.

Typically, the modified strain of microalgae has a greater than 10% reduction in the chitin or chitosan content compared to a chitin or chitosan content of a corresponding parent strain grown under same conditions, wherein the microalgae is typically selected from the Chlorellaceae taxonomic family of green algae of which notable genera include the true Chlorellae, such as but not limited to Chlorella vulgaris, or Chlorella sorokiniana in addition to other species including, but not limited to Parachlorella kessleri, Auxenochlorella protothecoides, Auxenochlorella pyrenoidosa, or Heterochlorella luteoviridis.

The modified strain of Chlorella vulgaris is obtained from the parent strain of Chlorella vulgaris, by performing mutagenesis. The mutagenesis may be performed by exposure of the parent strain of Chlorella vulgaris to a sub-lethal quantity of a mutagenic chemical for a specific time. Optionally, the specific time for treatment with the mutagenic chemical is 1 to 120 minutes. Typical mutagenic chemicals include alkylating agents such as ethyl methanesulphonate (EMS), methyl methanesulphonate (MMS), N-methyl-N-nitrosourea (NMU), N-methyl-N′-nitro-N-nitrosoguanidine (MNNG) and the like. The sub-lethal quantity of the mutagenic chemical is defined as the amount or quantity of the mutagenic chemical that results in less than 100% kill of the parent strain of Chlorella vulgaris in a given time. Alternatively, the mutagenesis may be performed by exposure of the parent strain of Chlorella vulgaris to a physical mutagen, wherein the physical mutagen comprises at least one of: UV light, gamma rays, X-rays.

Mutagenesis and, in particular, the use of sub-lethal quantities of a mutagenic chemical according to the invention, results in modified strains of Chlorella vulgaris in which the overall chitin content of the strain is the result of a stable genetic mutation. The same applies to other Chlorella microalgae species.

Without wanting to be bound by theory, it is believed that mutagenesis alters the primary amino acid sequence of chitin synthase, thereby affecting the activity of chitin synthase enzyme in the modified strains and further generations of the strains. Mutagenesis may also affect the biosynthesis of N-acetylglucosamine, the metabolic flux of N-acetylglucosamine in the biosynthesis of chitin, the biosynthesis of a chitinase or related enzyme class, or regulation of the same.

Optionally, mutagenesis alters the primary amino acid sequence of an ABC transporter or an aquaporin exhibiting a pleiotropic effect on chitin biosynthesis. Alternatively, mutagenesis alters a protein subunit functioning as a guanine nucleotide-binding protein with a role in ubiquitination or deubiquitination within chitin biosynthesis that may influence the steady-state activity of a chitin synthase enzyme based upon its residence time in the plasma membrane or ability to engage with accessory proteins involved in biosynthesis and/or translocation of the final product to the cell surface.

In addition to the low chitin content, which is linked to strain digestibility and nutrient bioavailability, the modified strain of Chlorella vulgaris or other Chlorella microalgae species of the invention may have one or more additional desirable phenotypes as a result of further stable genetic mutations. Such desirable phenotypes may be, for example, associated with organoleptic properties of the strain (e.g. colour, flavour, smell), content of other components such as, but not limited to, proteins, pigments (such as lutein and chlorophyll), micro- and macronutrients (for example potassium, sodium and nitrogen), minerals, vitamins, carbohydrates, fatty acids, antioxidants, glycoproteins, glycerols, phytochemicals (such as flavonoids and tannins), and dietary fibre content.

In another aspect of the invention, in addition to having a reduced chitin content in the cell wall, the algal cell may also have very low chlorophyll content. In a preferred aspect, the modified strain of Chlorella vulgaris has a chlorophyll content in a range of 0.25 to 0.50 mg/g dry cell weight, 0.10 to 0.25 mg/g dry cell weight or 0.001 to 0.1 mg/g dry cell weight. A low chlorophyll content avoids the common problem of undesirable organoleptic properties associated with wild-type microalgae biomass in general when used as a food or beverage ingredient. Optionally, the modified strain of Chlorella vulgaris has at least one of a white, cream, pale yellow, yellow, pale green, golden, orange, brown, pink, red or lime colour.

The modified strains of the invention can be mixotrophs or heterotrophs. In a preferred embodiment, the modified strains of the invention are cultivated in the dark (i.e. are competent heterotrophs and they are cultivable solely on an organic carbon energy source, in the absence of light). Beneficially, such heterotrophic growth allows large scale economical production of the modified strains as a result of the superior growth rate and biomass yield that can be produced in proven existing plant designs, when compared to photoautotrophic or mixotrophic methods of microalgal cultivation.

Typically, the modified strain of Chlorella vulgaris is cultivated at a specific temperature, preferably ranging from 20 to 35° C. and more preferably above 28° C., for a predefined period of time optionally without the presence of light, i.e. in the dark or absence of light, and in the presence of an organic carbon energy source such as for example glucose or acetate.

A preferred aspect of the invention is a non-genetically modified and non-transgenic, chitin-deficient strain of Chlorella vulgaris (having a chitin content less than 4.8 mg/g dry cell weight) which is a competent heterotroph. The strain may additionally be a chlorophyll-deficient strain.

The non-genetically modified and non-transgenic Chlorella vulgaris biomass is suitable for direct incorporation into food products, whole or as an ingredient. Food products include, but are not limited to, bakery products, microalgae flour, pasta, rice, breakfast cereals, cereal bars, confections, sauces, soups, dairy substitutes, frozen desserts, ice creams, yoghurts, smoothies, creams, spreads, salad dressings, mayonnaises, food garnishing and seasoning, candies, gums, jellies, vape liquid, beverages, snacks.

Advantageously, they are suitable as food ingredients in vegan products. Another advantage of the algal biomass of the invention is that, due to the low chitin content and therefore high digestibility, they may be incorporated into food products as whole or as an ingredient without the need for physical downstream processes such as pulverizing, milling, breaking, grinding, cracking. This reduces the energy intensity of the manufacturing process, thereby lowering the overall CO₂ footprint, creating a more sustainable, lower environmental impact route to the desirable ingredient.

The Chlorella vulgaris biomass of the invention is a suitable ingredient in the production of texturized vegetable protein (TVP) or similar meat analogue or meat extender which are products typically produced by extrusion.

Other uses include nutraceuticals, pharmaceuticals (including vaccines, various bioactives and delivery routes for other recombinant proteins and enzymes), nutritional supplements (for example, nutritional supplements, hormone tablets, digestive capsules, tablets, powders, oils and the like) and animal feed. Additionally, the other uses of the algal biomass include cosmetics (for example, in lipsticks, powders, creams, exfoliants, facial packs, and so forth), personal care compositions and personal care devices (for example toothpastes, mouthwash, hand-wash, body-wash, body soaps, shampoos, oils, sun-creams, after-sun creams, sunblock and so forth), colourants.

According to one aspect, an embodiment of the present disclosure provides a modified strain of Chlorella vulgaris with a chitin content of less than 4.8 mg/g dry cell weight and a chlorophyll content in a range of 0.001 to 0.5 mg/g dry cell weight. Preferably the chitin content is in a range of 0.001 to 4.8 mg/g dry cell weight. The chitin content may be in a range from 0.001 to 4.5 mg/g dry cell weight, 0.001 to 4.0 mg/g dry cell weight, 0.001 to 3.5 mg/g dry cell weight or 0.001 to 3.0 mg/g dry cell weight. Preferably, the chitin content is in a range 0.001 to 2.5 mg/g dry cell weight. More preferably, the chitin content is below 2.5 mg/g dry cell weight.

According to another aspect, in addition to the reduced chitin content, the modified strain of Chlorella vulgaris of the invention has a chlorophyll content of 0.25 to 0.50 mg/g dry cell weight, preferably 0.10 to 0.25 mg/g dry cell weight, and most preferably 0.001 to 0.1 mg/g dry cell weight.

When the modified strain of Chlorella vulgaris or another Chlorella microalgae species has substantially reduced chlorophyll content in addition to reduced chitin content, the product is particularly suitable for use as a food ingredient. The product not only avoids digestibility issues and may avoid reduced nutrient availability but has the additional advantages of not having the unpleasant colour, smell and/or taste associated with chlorophyll. Additionally, beneficially, such chlorophyll-deficient strains do not produce pheophorbide—a plant pigment, chlorophyll precursor, and breakdown product of chlorophyll which is known to cause skin irritation and photosensitive dermatitis, and which is known to accumulate in improperly treated and stored green algal products.

Notably, a modified strain of Chlorella vulgaris or another Chlorella microalgae species according to the invention is genetically stable and can be grown in a broad range of process conditions, ranging from optimal to stressful conditions, over time and not just limited to the use of light (sunlight or artificial light).

Another aspect of the invention is an optimized method of cultivation of said strain following mutagenesis. The modified strain of Chlorella vulgaris is obtained from a parent strain of Chlorella vulgaris or another Chlorella microalgae species by performing mutagenesis of the parent strain. The parent strain may be a wild-type strain of the algae or a variation of the wild-type strain. The variation of the wild-type strain may be a genetic mutant.

Typically, the mutagen is chemical or physical. Preferably the mutagen is a mutagenic chemical. More preferably, the mutagenic chemical is an alkylating agent. This is advantageous because chemical mutagenesis using alkylating agents for plant breeding, for human consumption, is not considered to produce Genetically Modified Organisms (GMOs) as defined by the current EU legislation. This is due to a safe and extensive history of successfully using this technique to establish improved plants. Mutagenesis may also be performed by exposing the parent strain of Chlorella vulgaris or another Chlorella microalgae species to a physical mutagen such as at least one of: UV light, gamma rays, X-rays.

After mutagenesis, chitin-deficient variants of the parent strain of Chlorella vulgaris or another Chlorella microalgae species are isolated. Isolation of suitable variants may be performed by any means known to the skilled person. The use of calcofluor white staining of the cells, flow cytometry or a combination thereof may be preferred. The method further comprises selecting healthy (or viable) cells of the modified strain of Chlorella vulgaris or Chlorella microalgae species. The healthy cells of the modified strain of Chlorella vulgaris or another Chlorella microalgae species refer to cells exhibiting desirable phenotype evident from a reduced calcofluor white staining, for example.

Mutagenesis and strain selection may be performed more than once. Performing several rounds of mutagenesis and strain selection also allows the production of modified strains of Chlorella vulgaris or another Chlorella microalgae species with desirable phenotypes, preferably a combination of such phenotypes. The method may further comprise selecting healthy cells of the modified strain of Chlorella vulgaris or another Chlorella microalgae species based on desired phenotypes. Desirable phenotypes may include amongst others and in addition to reduced chitin content, low chlorophyll content, desirable colours, a pigment content, a protein content or improved tolerance to process conditions.

The methods of the invention relate to Chlorella vulgaris and also to other Chlorella microalgae species.

The modified strain of Chlorella vulgaris is obtained after cultivation under heterotrophic growth mode. It is typically obtained after cultivation at a specific temperature ranging from 20 to 35° C., preferably above 28° C., for a predefined period of time ranging from 1 to 3 weeks without the presence of light, i.e. in the dark or in the absence of light, and in the presence of an organic carbon energy source such as for example glucose or acetate.

The identification of a modified strain of Chlorella vulgaris of the invention comprises sorting or screening the cells by any suitable technique, such as by using flow cytometry. The chitin deficient modified strain of Chlorella vulgaris may for example be selected based upon a calcofluor white fluorescence signal obtained on cell sorting by flow cytometry. The modified strain of Chlorella vulgaris may be further selected for example based on a desirable pigment or protein content, wherein the desirable pigment or protein content is based upon a relative signal obtained on cell sorting by flow cytometry. The use of flow cytometry provides the advantages of examining thousands of cells per second and in real time and processing quantifiable data over a computer coupled to a flow cytometer. Furthermore, flow cytometry helps in cell counting, cell sorting, determining cell characteristics and function and detecting microorganisms.

The method further comprises selecting healthy cells (or filtering out unhealthy cells) of the modified strain of Chlorella vulgaris, preferably by cultivation under non-permissive or stressful conditions.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF DRAWINGS

The invention is illustrated by the following figures, in which:

FIG. 1 is an illustration of steps of a method of producing a modified strain of Chlorella vulgaris having a chitin content of less than 4.8 mg/g dry cell weight;

FIG. 2 shows comparison of chitin content of parent strain (wild-type) and modified strains (p4-102, p5-94, p5-39 and p5-59) of Chlorella vulgaris represented as g/g DCW;

FIG. 3 shows comparison of chitin content of parent strain (wild-type) and modified strains (p4-102, p5-94, p5-39 and p5-59) of Chlorella vulgaris represented as % DCW;

FIG. 4 is a schematic illustration of a flow cytometry method to identify and isolate subpopulations of cells based on chitin content, following staining with calcofluor white;

FIG. 5 shows chitin content in modified strains of Chlorella vulgaris as compared to chitin content in parent (wild-type) strain of Chlorella vulgaris cultivated under the same conditions represented as fluorescence in a confocal calcofluor white staining;

FIG. 6 shows a comparison of protein digestibility-corrected amino acid score (PDCAAS) for a range of protein sources; and

FIG. 7 is an illustration of chitin biosynthesis in parent (or wild-type) strain of Chlorella vulgaris 4TC3/16.

### DETAILED DESCRIPTION

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

It is understood that the invention, methods and approach described herein also apply to the identification and isolation of chitin deficient modified strains of other green algae that are haploid genetically and possess a chitin-like polysaccharide or glucosamine component within their cell walls. Such algae include, but are not limited to: genus Chlorella or Parachlorella, such as Chlorella vulgaris, Chlorella sorokiniana and Parachlorella kessleri (Takeda 1991; DOI: DOI:10.1111/j.0022-3646.1991.00224.x) or algae which are closely phylogenetically related, but may have been previously taxonomically misassigned; including strains of Chlorella protothecoides (Auxenochlorella protothecoides), Chlorella pyrenoidosa (putatively Auxenochlorella pyrenoidosa), Chloroidium ellispoideum and Chloroidium saccharophilum (Baudelet et al. 2017; DOI: 10.1016/j.algal.2017.04.008).

In overview, embodiments of the present disclosure are concerned with a modified strain of Chlorella vulgaris having a reduced chitin content, specifically, a chitin content of less than 4.8 mg/g dry cell weight. The overall chitin content of the modified strain is lower than the chitin content of a parent strain of Chlorella vulgaris grown under the same conditions. Furthermore, embodiments of the present disclosure are concerned with a method of producing the modified strains of Chlorella vulgaris having a reduced chitin content, compositions comprising algae biomass derived from such strains and to their use as food ingredients amongst other applications.

The invention is further described by reference to the following definitions used herein.

The term “Chlorella vulgaris” as used herein, refers to a species of single-cell aquatic plant, termed microalgae, falling under Division “Chlorophyta” within the plant taxonomic Kingdom. The full taxonomic assignment is: Biota Plantae (Kingdom) Viridiplantae (Subkingdom) Chlorophyta (Phylum (Division)) Chlorophytina (Subphylum (Subdivision)) Trebouxiophyceae (Class) Chlorellales (Order) Chlorellaceae (Family) Chlorella (Genus) Chlorella vulgaris (Species). The microalgae are photosynthetic organisms that grow in diverse habitats ranging from regions of varying hardness of growth medium (such as soil or water), humidity, salinity, light-access, and temperature conditions, such as land, rivers, ponds, lakes, sea, brackish water, wastewater and the like.

The term “Chlorella microalgae species” as used herein, refers to a species of single-cell aquatic plant, termed microalgae, falling under Division “Chlorophyta” within the plant taxonomic Kingdom. The full taxonomic assignment is: Biota Plantae (Kingdom) Viridiplantae (Subkingdom) Chlorophyta (Phylum (Division)) Chlorophytina (Subphylum (Subdivision)) Trebouxiophyceae (Class) Chlorellales (Order) Chlorellaceae (Family) Chlorella (Genus), and especially a chitin-containing species.

The term “chitin” as used herein refers to a structural polysaccharide containing nitrogen and glucosamine and includes chitin, chitin-like polysaccharide and chitosan. Chitin is synthesized, from monomer units N-acetyl-D-glucosamine linked by a β-(1,4) covalent linkage, by a chitin synthase enzyme. Chitin is insoluble in most organic solvents such as water and dilute acids and, as a result, imparts rigidity to the cell walls. Chitin is a primary component of cell walls of a varied group of organisms, such as yeast, fungi, molluscs, arthropods (for example insects and crustaceans), nematodes, and marine animals (for example fishes and invertebrates). Interestingly, chitin is not a typical component of the cell walls in green algae and the presence of chitin is one of the genus-defining characteristics of Chlorella species.

The term “wild-type strain” (or wild-type) refers to a typical form of an organism as it occurs in nature. Specifically, the wild-type is a typical form of an organism of a species comprising a set of genes characteristic to a naturally existing organisms of that species, i.e. comprising normal occurrence of a gene at a locus, and exhibiting the associated phenotypes thereof. The wild-type strain of Chlorella vulgaris can be obtained from culture collections or its usual dwelling sites such as land, rivers, ponds, lakes, brackish water, wastewater and the like. The naturally existing wild-type strain of Chlorella vulgaris is able to grow autotrophically by performing photosynthesis. During the process of photosynthesis, the wild-type strain of Chlorella vulgaris utilizes sunlight, carbon dioxide, water and a few nutrients to produce a biomass of alga. However, the wild-type strain of Chlorella vulgaris can also be cultivated using heterotrophic and/or mixotrophic growth modes. Wild-type strains of Chlorella vulgaris are haploid in their normal growth phase, i.e. have only one copy of the genome, thereby making Chlorella vulgaris particularly amenable to a phenotypic trait improvement approach using genetics as, for some traits, a single genetic change could yield the desired phenotype. Furthermore, being haploid, these variant or improved strains are likely to be genetically stable as there is essentially no capacity of the mutant strain to easily correct or revert to the wild-type state; moreover, there is no other genetic copy of the DNA that can act as a correction template to facilitate this process. The wild-type strains of Chlorella vulgaris are associated with a dark-green colour, a specific smell (such as aquatic, fish-like, earthy or mouldy smell), an unpleasant taste, in addition to a cell wall comprising an alkali soluble hemicellulose fraction, and a residue fraction; the rigid wall. The wild-type strain of Chlorella vulgaris has high chitin content that contributes to its rigid cell wall, with this fraction typically comprising between 60 and 66% of the overall cell wall—this level generally remains constant throughout different stages of growth (as described by Abo-Shady et al. 1993; DOI: 10.1007/BF02928041). The wild-type strain of other Chlorella microalgae species can be obtained from culture collections or its usual dwelling sites such as land, rivers, ponds, lakes, brackish water, wastewater and the like.

The term “parent strain” as used herein, refers to a donor organism that donates its DNA to an offspring or daughter cell thereof. Specifically, Chlorella vulgaris reproduces asexually by multiple fission, with the basic rule that one mother cell reproduces its DNA synchronously to produce at least two daughter cells per division event (or burst). Occasionally, a division burst may comprise four, eight and rarely, sixteen daughter cells (Mandalam and Palsson 1997; DOI 10.1023/A:1018310008826). The number of Chlorella vulgaris daughter cells produced per division burst is thought to be modifiable by environmental factors such as light and temperature—being as they directly affect growth rate, and consequently, the coordination between DNA replication and division events in the cell cycle (Bišová and Zachleder 2014; DOI: 10.1093/jxb/ert466). Given this asexual method of whole genome reproduction and inheritance, it can be understood that Chlorella vulgaris strains exhibit an extremely high degree of genetic stability between generations. Further, in the context of a mutagenesis campaign, the parent strain may be a wild-type strain of Chlorella vulgaris or a variation (i.e. a genetic variant) of the wild-type strain of Chlorella vulgaris. The term “parental strain” therefore may also refer to a genetic variant or subtype of Chlorella vulgaris, preferably a previous generation.

It will be appreciated that a variation of the wild-type strain of Chlorella vulgaris or other Chlorella microalgae species differs from the parent strain (namely, the wild-type strain) only by the mutated gene(s) (and in some cases closely liked genes). Such modified strains of Chlorella vulgaris are valuable in understanding the effect of a single or multiple gene mutations in the organism. The variation of the wild-type strain of Chlorella vulgaris may be a genetic mutant.

The term “mutagenesis” as used herein, relates to a technique of inducing mutations by artificially exposing the organism to mutagens using laboratory procedures. Mutagens have the effect of increasing the frequency of genetic mutation over and above the natural frequency of spontaneously occurring mutations.

The term “alkylating agents” as used herein, refers to classes of alkylating agents functioning as mutagens that include but are not limited to: sulphur mustards, nitrogen mustards, epoxides, ethylene imines, alkyl alkanesulphonates, dialkyl sulphates, beta-lactones, diazo compounds and nitroso compounds. Examples of such alkylating agents from each of these respective classes include: mustard gas, nitrogen mustard (HN2), ethylene oxide (EO), diepoxybutane (DEB), ethyleneimine (EI), triethylenemelamine (TEM), ethyl methanesuIphonate (EMS) and methyl methansulphonate (MMS), diethylsulphate (DES), beta-propiolactone, diazomethane, N-Nitroso-N-methylurea (NMU) and N-methyl-N′-nitro-N-nitrosoguanidine (NG or NTG or MNNG) (as described by Auerbach 1976; DOI: 10.1007/978-1-4899-3103-0_16).

The term “chlorophyll” as used herein refers to a group of green pigments contained in cells of green plants. Chlorophyll is essential for photosynthesis and allows photosynthetic organisms to absorb energy from sunlight (absorbing blue and red lights and reflecting green light from the visible region of the electromagnetic spectrum). It will be appreciated that the chlorophyll content is associated with at least one of: chlorophyll a (α-chlorophyll or Chl-a) and/or chlorophyll b (β-chlorophyll or Chl-b). Chlorophyll a is a primary photosynthetic pigment, which participates directly in the light-driven reactions of photosynthesis, while chlorophyll b is an accessory pigment operable to collect energy primarily from blue wavelengths of sunlight and pass it on to chlorophyll a. Moreover, the chlorophyll content is influenced strongly by cultivation conditions, in particular the absence or presence of light. In the dark, chlorophyll content is naturally suppressed.

The term “lutein” refers to a primary xanthophyll (carotenoid) in green microalgae such as Chlorella vulgaris. Lutein enables the microalgae to absorb blue light and reflect yellow or orange-red light from the visible region of the electromagnetic spectrum. Lutein functions as a light energy modulator in Chlorella vulgaris and serves as a non-photochemical quenching agent that protects cells of the Chlorella vulgaris from photochemical damage caused by high intensity of light during photosynthesis. Moreover, the lutein content in an organism is genetically determined and regulated by growth conditions, including but not limited to temperature, pH of growth medium, exposure to light, nitrogen content in the growth medium or atmosphere, salinity of growth medium, rate of growth and so forth.

The term “Protein digestibility-corrected amino acid score (PDCAAS)” refers to a standard method for evaluating quality of a protein of a specific food. PDCAAS rating has been adopted by the Food and Drug Administration (FDA, US) and the Food and Agricultural Organization (FAO, WHO) as “the preferred ‘best’” assay for determining protein quality. Specifically, PDCAAS assay combines the protein digestibility with a protein quality based on the amino acid requirements of humans and expresses the result as an absolute percentage score value. For example, a score of 100% (or 1.0) refers to a measure of a protein's ability to provide adequate levels, such as 100% in this case, of essential amino acids per unit protein for human needs. The essential amino acids include histidine, isoleucine, leucine, lysine, methionine, cystine, phenylalanine, tyrosine, threonine, tryptophan and valine. In the present disclosure, the PDCAAS is calculated using the Dumas method for protein assay with a nitrogen to protein conversion factor of 6.25 to normalize the calculated score to other protein sources for comparison.

The term “genetically stable” as used herein, refers to a characteristic of a species or a strain/isolate to resist changes and maintain its genotype over multiple generations or cell divisions, ideally hundreds to thousands.

The term “algae biomass” as used herein refers to biomass derived from algae (microalgae or macroalgae) that is cultivated heterotrophically. Optionally, the algae biomass can be obtained from the modified strain of Chlorella vulgaris under current good manufacturing practice (cGMP) conditions.

The term “food” refers to an edible product that can be directly or indirectly (such as, subsequent to preparation) consumed by humans and/or animals. The term “food ingredient” refers to a substance incorporated into food during one of: production, processing, treatment, packaging, transportation, distribution, preservation, storage and so forth of food. Optionally, the food ingredients are incorporated into the food to improve and/or maintain freshness, nutritional value, appearance, texture, taste and safety of the food.

The term “microalgae flour” is used to refer to an edible composition comprising a plurality of particles of algae biomass. Optionally, the plurality of particles of algae biomass is any one of: whole cells, lysed cells or a mixture thereof. More optionally, the microalgae flour comprises one or more of significant digestible proteins, dietary fibre content, associated water binding attributes, healthy oil delivering attributes, spices, herbs, a flow agent, an antioxidant and so forth. It may be appreciated that the microalgae flour lacks visible oil and is preferably in a powdered form. The microalgae flour can be produced under current Good Manufacturing Practice (cGMP) conditions.

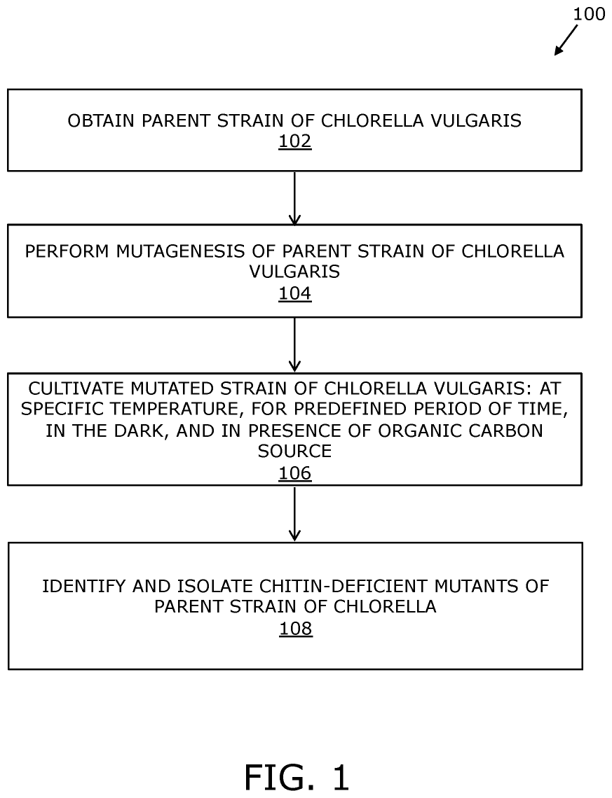

FIG. 1 shows steps of a method 100 of producing a modified strain of Chlorella vulgaris having a chitin content of less than 4.8 mg/g dry cell weight, in accordance with an embodiment of the present disclosure. Typically, the cell wall of the Chlorella vulgaris species is composed of a hemicellulose component and a glucosamine component. The glucosamine component constitutes between 60 and 66% of the overall cell wall and this level generally remains constant throughout different stages of growth (as described by Abo-Shady et al. 1993; DOI: 10.1007/BF02928041). The glucosamine component of the cell wall of Chlorella vulgaris is essentially chitin (poly-β-(1,4)-N-acetyl-D-glucosamine) or a chitin-like polysaccharide (such as chitosan (poly-β-(1,4)-2-amino-2-deoxy-D-glucose), produced by diacetylation of chitin by a chitin deacetylase enzyme).

At a step 102, the parent strain of Chlorella vulgaris is obtained. The obtained parent strain of Chlorella vulgaris is genetically defined as Chlorella vulgaris using PCR amplification, sequencing and alignment of the genetic material with a reference sequence. Examples of useful genetic sequencing targets for the purpose of taxonomic identification of Chlorella vulgaris include, but are not limited to: 18S rRNA gene sequence, the internally transcribed spacer (ITS) regions between the 18S rRNA gene, 5.8S rRNA gene and the 28S rRNA gene sequence. Such regions have been used extensively for intra and inter genus phylogenetic analysis of the Chlorellaceae (green algae) family (Huss et al. 1999; DOI: 10.1046/j.1529-8817.1999.3530587.x, Krienitz et al., 2015; DOI: 10.1016/j.tplants.2014.11.005, Darienko and Pröschold 2015; DOI: 10.1111/jpy.12279, and Heeg and Wolf 2015; DOI: 10.1016/j.plgene.2015.08.001). Other statistics and additional sequences derived from whole genome sequencing is another method for strain identification. In an example, the pair-wise sequence similarities between the sequence amplified by PCR from Chlorella vulgaris 4TC3/16 and a non-redundant sequence collection (GenBank, EMBL, DDBJ, PDB and RefSeq sequences) are calculated using the Basic Local Alignment Search Tool. Identity is calculated with the maximum length of the shortest sequence with another sequence. While the second closest species based on the identity score, Chlorella sorokiniana would only reach 99.55% identity, the 18S rRNA gene sequence of Chlorella vulgaris 4TC3/16 showed 99.9% identity with the 18S rRNA gene sequence of other Chlorella vulgaris strains over the full-length (1800 bp) 18S rRNA gene sequence. The identification is further confirmed using ITS2 sequencing. This excludes 4TC3/16 to belong to any other species of Chlorella, other than Chlorella vulgaris.

At a step 104, mutagenesis of the parent strain of Chlorella vulgaris is performed. The modified strain of Chlorella vulgaris is obtained from the parent strain of Chlorella vulgaris by performing mutagenesis of the parent strain of Chlorella vulgaris. The parent strain of Chlorella vulgaris is subjected to mutagenesis in order to produce mutated, variant strains of Chlorella vulgaris exhibiting a different phenotype, such as reduced chitin content, colour, and so on, from that exhibited by the parent strain of Chlorella vulgaris.

Typically, the mutagenesis of the parent strain of Chlorella vulgaris is performed by exposure of the parent strain of Chlorella vulgaris to a sub-lethal quantity of a mutagenic chemical for a specific time. Optionally, the specific time for treatment with the mutagenic chemical is 1 to 120 minutes.

Optionally, the mutagenic chemical is an alkylating agent. Generally, the alkylating agents transfer alkyl groups (such as methyl or ethyl group) to macromolecules (such as bases, or the backbone phosphate groups of the nucleic acids) under physiological conditions. Typically, the alkyl group acts on nucleophilic sites of the macromolecule, for example, nitrogen or oxygen nucleophiles in DNA (as described by Gates 2009; DOI: 10.1021/tx900242k). Such transfers result in alkylation of bases (for example guanine) and subsequent mispairing of said base during DNA replication (with for example, thymine instead of cytosine). It will be appreciated that the alkylating agents function similar to EMS in producing mutations in the genetic makeup of the organism exposed thereto. Repeated replication of such mispaired DNA can result in a transition mutation, wherein original G:C base pairs change to A:T base pairs, thereby changing the genetic makeup of the organism. In such case, the replication of such mutated DNA may create heritable missense mutations or nonsense mutations within coding sequences or impacting gene expression or gene function by compromising regulatory sequence functionality including RNA splice-site mutations or promoter or other regulatory sequence mutations. Beneficially, the use of alkylating agents as mutagenic chemicals for plant breeding, for human consumption, is not considered to produce Genetically Modified Organism (GMOs) as defined by the current EU legislation, and is therefore acceptable for further applications in various industries, such as food, health, biotechnology and biofuels.

As mentioned, hereinabove, a sub-lethal quantity of the mutagenic chemical may be used for performing the mutagenesis. Optionally, the sub-lethal quantity of the mutagenic chemical is defined as the quantity that results in less than 100% lethality (or kill) of the parent strain of Chlorella vulgaris in a given time (namely, exposure time). More optionally, the quantity of the mutagen (or mutagen dose) is defined as a concentration of the mutagen multiplied by an exposure time. Specifically, the sub-lethal quantity of the mutagen is obtained by altering the mutagen concentration, the exposure time, or a combination of both, for example. Optionally, the sub-lethal quantity of the mutagenic chemical ranges between 0.1 to 2.0 M. The sub-lethal quantity of the mutagenic chemical may be for example 0.1, 0.2, 0.3, 0.4, 0.5, 0.6, 0.7, 0.8, 0.9, 1.0, 1.1, 1.2, 1.3, 1.4, 1.5, 1.6, 1.7, 1.8 or 1.9 M up to 00.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 M. In an example, the sub-lethal quantity is 0.2 M of EMS that is non-lethal to the Chlorella vulgaris species. In another example, the sub-lethal quantity is 0.2 M of MMS that produces a 20% lethality to the Chlorella vulgaris species (referred to as “mutagen kill” hereafter). In yet another example, the sub-lethal quantity is 0.8 M of EMS that produces a 40% mutagen kill. In still another example, the sub-lethal quantity is 0.8 M of MMS that produces a 60% mutagen kill. The exposure time to a given concentration of mutagen also influences its lethality. In this regard, the mutagenesis of the parent strain of Chlorella vulgaris may be performed by exposure of the parent strain of Chlorella vulgaris to a 1.0 M dose of EMS for an exposure time of 1 minute or a dose of EMS above 1.0 M for exposure time of a shorter period, for example 30 seconds, to produce a 50% lethality to the Chlorella vulgaris species, for example. This process, combining mutagen concentration and exposure time to said mutagen, results in a kill rate which acts as a proxy for mutation frequency. Consequently, surviving cells of said process have one or more mutations within their genomes. Collectively, such cells comprise a pool (or library) of mutations from which can be selected desirable variant strains using a suitable method.

It will be appreciated that the quantity of the mutagenic chemical used for performing the mutagenesis, combined with the exposure time, can determine the amount of mutation undergone by the organism. Furthermore, heavily mutagenized cells of the organism accumulate multiple mutations of genetic material, which are often deleterious to the viability or overall fitness of the mutagenized strain as assessed using standard growth performance assays, for example. This is of particular importance in a haploid organism, where only a single copy of each gene is present. It is common that multiple mutations occur within the genomes of mutagenized strains. Use of a high quantity of alkylating agent for performing the mutagenesis may result in point mutations that create aberrations in the mutated strain of Chlorella vulgaris as compared to the parent strain of Chlorella vulgaris or may result in death of the mutated strain of Chlorella vulgaris. Therefore, using a sub-lethal or non-lethal quantity (0.2 to 2.0 M) of the mutagenic chemical, such as alkylating agents, for a specific time, enables generation of desired phenotypes while preventing or minimising accumulation of undesirable traits that might reduce overall strain fitness, hamper growth, or result in death of the organism. Furthermore, cultivation of the cells that have been exposed to mutagenic chemical at a higher than optimal cultivation temperature acts as a ‘stress’ filter such that only the more robust strains—where accumulated mutations have not produced a weakened or crippled organism can produce colonies on agar or viable daughter cells identified through a screen such as flow cytometry. As a result of cultivation of mutagenized cells at such elevated temperature or temperatures, fewer overall cells grow but those that do grow are more biologically and genetically fit with regard to growth and/or biomass production. Hence, those strains with reduced chitin content that grow under these conditions and are scored based upon initial chitin content should also be expected to be more robust with regard to application within an ultimate scalable commercially-relevant bioprocess.

Mutagen kill can be used as a proxy to estimate or model mutation frequency achieved per quantity of mutagen in a defined period of exposure time (namely dose). It is important to achieve an optimal mutation frequency for the least number of cells killed, to ensure the generation of a high-quality mutant library. It will be appreciated that such a high-quality mutant library would preferably comprise a highly diverse number of mutations, spread across a large number of viable cells, sufficient to ensure that each individual mutant strain contains a sufficiently low number of mutations to enable isolation of advantageous genotypes, without aliasing, while also avoiding the potentially deleterious effects of multiple mutations on growth performance and other quality metrics, such as biochemical composition and the like. Optionally, the degree of mutagen kill may be measured by determining cell viability using a conventional quantification technique (for example, viable counts, viability staining, flow cytometry, and the like) known in the art. In some organisms, resistance to a growth inhibitor can be based on a single genetic (point) mutation, for example antibiotics such as aminoglycosides, quinolones, rifampicin, pyrazinamide, and isoniazid. In the context of the present invention, the mutation frequency of a mutagen dose can be directly quantified by using any method known in the art.

It will be appreciated that it is possible to saturate the mutagen dose; whereby continuing to increase either the concentration of mutagen used, or the exposure time, or a combination of both, can result in diminishing returns on cell viability, with no corresponding increase in mutation frequency. Typically, optimal mutagen dose is determined empirically for a specific species of an organism, and varies from organism to organism. Similarly, different mutagens have different mechanisms of action, and an optimal dosing strategy (i.e. relative concentration multiplied by time) using a mutagen for the target organism can be determined for each. Besides determining the dosing strategy, identification of a suitable mutagen is equally essential as each mutagen class has a specific mechanism of action that directly affects the diversity of mutations generated in the target organism which, thereby, also impacts the utility of the resulting mutant library. It follows, therefore, that some mutagens by their action are not suitable for a particular target organism, for example where they produce an unsuitably high or unsuitably low mutation frequency (or mutagen kill) even at titrated doses to be practically useful—this is often determined empirically, especially in the case of proprietary organisms such as Chlorella vulgaris strain 4TC3/16.

Optionally, the mutagenesis is performed by exposure of the parent strain of Chlorella vulgaris to a physical mutagen, wherein the physical mutagen comprises at least one of: UV light, gamma rays, X-rays. These mutagens cause changes in the genotype of the parent strain of Chlorella vulgaris to result in the mutated strain of Chlorella vulgaris. In such an instance, as an alternative to performing the mutagenesis of the parent strain of Chlorella vulgaris by exposure to the mutagenic chemicals, mutagenesis by exposure to physical mutagens can be performed to obtain the mutated strain of Chlorella vulgaris.

Optionally, the chitin content of the modified strain of Chlorella vulgaris is a result of a stable genetic mutation. To clarify, the difference in the chitin content of the modified strain of Chlorella vulgaris as compared to the chitin content of the parent strain of Chlorella vulgaris is the direct result of mutations in the genome of the modified strain of Chlorella vulgaris as a result of the exposure to mutagens, preferably mutagenic chemical, more preferably alkylating agent. The repeated cultivation of the strains in the same growth conditions, i.e. heterotrophic growth conditions, produces generations of the modified strain of Chlorella vulgaris with chitin content thereof comparable to the chitin content of the starting mutant strain thereof.

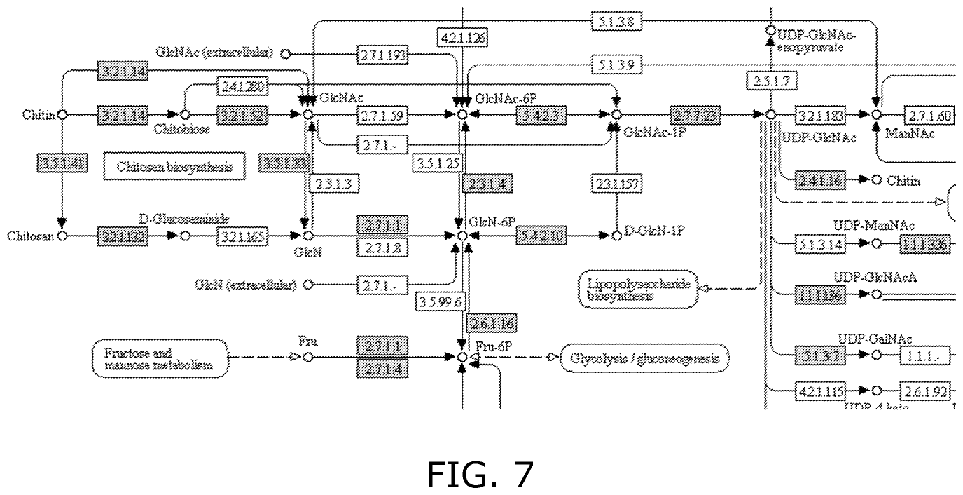

Moreover, the modified strain of Chlorella vulgaris has a stable genetic mutation affecting one or more of: a primary amino acid sequence of chitin synthase, a regulator of chitin synthase, biosynthesis of N-acetylglucosamine, a metabolic flux of N-acetylglucosamine in the biosynthesis of chitin, and biosynthesis of a chitinase or related enzyme class. As discussed above, chitin is synthesized by a chitin synthase enzyme. As a result of an alteration in the primary amino acid sequence of the enzyme chitin synthase, the regulation of chitin synthase enzyme is affected. Moreover, apart from chitin synthase, the biosynthesis of chitin in Chlorella vulgaris cell wall involves various other enzymes such as those involved in the formation of D-Glucosamine 6-phosphate (GlcN-6P), N-Acetyl-D-glucosamine 6-phosphate (GlcNAc-6P), N-Acetyl-alpha-D-glucosamine 1-phosphate (GlcNAc-1P), UDP-N-acetyl-alpha-D-glucosamine (UDP-GlcNAc) and so forth (as shown in FIG. 7). Therefore, a stable genetic mutation caused by the mutagenic chemical, preferably alkylating agent, targets the cell wall synthesis and produces a specific alteration in chitin biosynthesis pathway, such as in the chitin synthase enzyme, a gene encoding for the chitin synthase enzyme, or a pathway involving said enzyme or said gene.

Optionally, the modified strain of Chlorella vulgaris has a stable genetic mutation affecting the primary amino acid sequence of an ABC transporter resulting in a pleiotropic effect on chitin biosynthesis. ABC transporters might be serving a “cofactor” role in chitin biosynthesis potentially being involved in the translocation of the polymer across the cell membrane to accumulate on the outside of the cell to be incorporated into the cell wall. Specifically, the gene encoding a putative ABC transporter carries a mutation resulting in a change from Arginine 347 to Cysteine in the primary amino acid sequence (SEQ ID NO: 1). at the end of a predicted ABC transporter transmembrane region.

Optionally, the modified strain of Chlorella vulgaris has a stable genetic mutation affecting a protein subunit functioning as a guanine nucleotide-binding protein with a pleiotropic effect on chitin biosynthesis. Mutagenesis alters the primary amino acid sequence of a protein subunit functioning as a guanine nucleotide-binding protein. Based on homology modelling using PANTHER (http://www.pantherdb.org/) and eggNOG tools (http://eggnog5.embl.de), this protein may function in the regulation of protein monoubiquitination. Its most probable location is cytoplasmic. The genetic mutation (SEQ ID NO: 2) is localised at a splicing donor site preventing splicing of the mRNA after exon 9, (after amino acid 379) at the end of the WD-40 repeat domain. Consequently, mutation in this gene may result in a non-functional, truncated protein product or even a dominant negative acting truncated protein. Proteins with a WD-40 repeat domain carry out a very wide diversity of functions. However, the most probable function of this protein is in ubiquitination or deubiquitination. For instance, Q8TAF3 (https://www.uniprot.org/uniprot/Q8TAF3—WDR48_HUMAN) has similar structure (WD-40 repeat and DUF3337 domains) and has been shown to be a Regulator of deubiquitination complexes. There is evidence that ubiquitinination can regulate chitin synthase Chs3 post-translationally in yeast (Arcones et al. 2016; DOI: 10.1091/mbc.E16-04-0239). Moreover, the residence time at the cell surface of predicted CHS enzymes as well as activity of the enzymes themselves could be impacted by the ubiquitination state of the cell surface, thereby impacting the steady state amount of chitin that accumulates at the cell surface.

Optionally, the modified strain of Chlorella vulgaris has a stable genetic mutation affecting the primary amino acid sequence of an aquaporin or aquaglyceroporin-related protein resulting in a pleiotropic effect on chitin, or chitin-like polysaccharide composition or content of the cell wall. Specifically, the gene encoding said protein carries a mutation resulting in a change from Alanine 46 to Valine in the primary amino acid sequence (SEQ ID NO: 3), which is predicted to be located in the first major intrinsic protein domain.

At a step 106, the mutated strain of Chlorella vulgaris is cultivated: at a specific temperature, for a predefined period of time without the presence of light, and in the presence of an organic carbon source. Optionally, the selection is carried out under photoautotrophic conditions. More optionally, the selection is carried out under mixotrophic conditions; comprising both light and an organic carbon source of energy, such as glucose or acetate. Notably, algae such as Chlorella vulgaris can grow in conditions ranging from optimal to extreme and in varied habitats. Typically, the parent strain of Chlorella vulgaris is exposed to a mutagenic chemical (such as an alkylating agent) or optionally to a physical mutagen, at specific temperature conditions. Typically, the parent strain of Chlorella vulgaris is exposed to the mutagenic chemical or optionally, to a physical mutagen, for the predefined period of time. Such an exposure of the parent strain of Chlorella vulgaris at specific temperature conditions and for a predefined period of time enables production of modified (or mutated) strains of Chlorella vulgaris. Optionally, the specific temperature is in the range of 20 to 35° C., preferably in the range of 25 to 28° C., and most preferably above 28° C., and the predefined period of time for cultivation post-exposure is in a range of 1 to 5 weeks, and preferably 1 to 3 weeks. In an example, the parent strain of Chlorella vulgaris is exposed to the alkylating agent at 25° C. for 2 hours.

Preferably, the mutated strain of Chlorella vulgaris is obtained from a parent strain of Chlorella vulgaris that can be cultivated without presence of light. The term “without presence of light” as used herein refers to cultivating in the dark or in the absence of light. In such case, as an example, the petri dishes containing the sample of Chlorella vulgaris may be wrapped individually in a substantially opaque sheet, such as a foil, and then the wrapped-up petri dishes may be placed inside a cardboard box in the incubator. Other suitable ways of cultivating in the dark or without the presence of light can be used.

Optionally, the mutated strain of Chlorella vulgaris of the invention is obtained from a parent strain of Chlorella vulgaris cultivated in the presence of low light. It will be appreciated that the mutated strain can grow without the presence of light so long as there is an exogenous carbon source such as acetate or glucose but that some mutations and resulting strains may be rendered hyper-sensitive to light such that cultivation above very low light levels, for instance 5 micromoles/m²/second, has an inhibitory or toxic effect on growth. Optionally, the characteristics of the light used (e.g. intensity of light, wavelength (colour) of light and so forth) during mutagenesis, are defined. More optionally, the light intensity range, wavelength and quality may be, i.e. white LED, white fluorescent, daylight fluorescent, red LED, or mix of white and red or other LED, with light intensity values ranging from 5 micromoles/m²/s to 300 micromoles/m²/s, most preferably low light conditions comprise 2 to 25 micromoles/m²/s of white LED light.

Optionally, the mutated strain of Chlorella vulgaris of the invention is obtained from a parent strain of Chlorella vulgaris cultivated solely in the presence of light. The term “solelyin the presence of light” used herein refers to cultivating strains of Chlorella vulgaris under photoautotrophic growth mode, where light is the source of energy used to provide both reducing power (in the form of NADPH) and ATP (produced by photophosphorylation of ADP) in the fixation of CO2 into carbohydrate. In photoautotrophic growth mode, Chlorella vulgaris may be cultivated in a defined organic salt medium, without the addition of exogenous organic carbon, such as glucose or acetate. The medium may be liquid or comprise a solid agar. The light intensity range, wavelength and quality may be, i.e. white LED, white fluorescent, daylight fluorescent, red LED, or mix of white and red or other LED, with light intensity values ranging from 5 micromoles/m²/s to 300 micromoles/m²/s, most preferably 30 to 200 micromoles/m²/s of white LED light.

Optionally, the mutated strain of Chlorella vulgaris is obtained from a parent strain of Chlorella vulgaris cultivated using one or more of: a liquid or solid growth medium, including a fermentation medium containing an added carbon source such as glucose, or a mixotrophic growth medium containing acetate or a heterotrophic growth medium or an autotrophic growth medium whereby CO2 is used as the carbon source via a photosynthetic route and growth in the light. In an example, the mutated strain of Chlorella vulgaris is obtained from a parent strain of Chlorella vulgaris cultivated using a solid medium. Such a solid medium can be a regular agar plate. In such an instance, cells of the mutated strain of Chlorella vulgaris are inoculated on agar plates at an appropriate cell density to achieve a dense biomass growth on the surface of the agar plates. It is important to note that addition of a simple carbon source such as, but not limited to, glucose (dextrose), acetate or other simple carbon compound allows the cells to grow in the dark under heterotrophic growth mode or in low light under mixotrophic growth mode. The solid medium can be a high salt medium-glucose agar plate, wherein the high salt medium-glucose agar plate comprises: a growth medium such as High Salt Medium™ (HSM™), glucose (for example, 1% w/v) and agar.

In another example, the mutated strain of Chlorella vulgaris is cultivated using a liquid medium. Such a liquid medium can be at least one of TAP (Tris-Acetate-Phosphate), High Salt Medium™ (HSM™), glucose (for example, having consistency of 1% w/v) and so forth. The fermentation medium comprises a source of nitrogen (such as proteins or nitrate or, more usually, ammonium), minerals (including magnesium, phosphorus, potassium, sulphur, calcium, and iron), trace elements (zinc, cobalt, copper, boron, manganese, molybdenum), an optional pH buffer, a source of carbon and energy (such as glucose, acetate) and so forth. Optionally, the parent strain of Chlorella vulgaris is cultivated in a fermenter.

Optionally, the mutated strain of Chlorella vulgaris is cultivated under heterotrophic growth mode. For example, the mutated strain of Chlorella vulgaris is cultivated under heterotrophic growth mode without any presence of light (i.e. in the dark). In such an example, heterotrophic growth of the mutated strain of Chlorella vulgaris is achieved under suitable aseptic conditions. The heterotrophic growth can be carried out by growing the mutated strain of Chlorella vulgaris using a source of carbon and energy, such as glucose, without the presence of light. Alternatively, the mutated strain of Chlorella vulgaris is cultivated under mixotrophic growth mode with partial presence of light, such as by exposure of the mutated strain of Chlorella vulgaris to light for a limited time per day or at a minimally set light intensity. In such an example, the mixotrophic growth is performed by employing simultaneous use of different sources of energy for cultivating the mutated strain of Chlorella vulgaris. Alternatively, the mutated strain of Chlorella vulgaris is cultivated under autotrophic growth mode in the light with supply of air or a specific CO₂ supply to facilitate photosynthetic growth.

Furthermore, the Chlorella vulgaris uses different sources of energy, along with light, in different combinations for growth. Optionally, the modified strain of Chlorella vulgaris is a heterotroph. More optionally, the modified strain of Chlorella vulgaris is a heterotroph exhibiting improved tolerance to stress as a consequence of the selection strategy. Beneficially, more-tolerant heterotrophs are suitable for growth in stressful heterotrophic growth conditions without affecting the cell growth rate. In this context, a stressful condition is a growth parameter that falls outside the range of cultivation conditions which normally results in optimal growth. Examples of such a parameter include, but are not limited to: temperature, pH, osmolality, barometric pressure, media composition, agitation speed and the like. It will be appreciated that a stressful cultivation condition could result from a combination of one or more such parameters. Additionally, beneficially, such tolerant heterotrophs enable cultivation of said organism faster (i.e. due to faster growth rate) and in a large quantity (i.e. higher growth yield) under stressful or non-optimal cultivation conditions.

Optionally, the parent strain of Chlorella vulgaris is treated with a mutagen, preferably a mutagenic chemical, and most preferably an alkylating agent, and the variants (or mutated strains) are then selected based on a desirable phenotype, preferably reduced chitin content, after growth on solid medium.

At a step 108, the chitin-deficient mutants of the parent strain of Chlorella vulgaris are identified and isolated. Cells of the mutated strain of Chlorella vulgaris having a phenotype different from the parent strain of Chlorella vulgaris are identified as the modified strain of Chlorella vulgaris and subsequently isolated for further application thereof. For example, when the mutated strain of Chlorella vulgaris is cultivated using agar plates, colonies of the mutated strain of Chlorella vulgaris on the agar plates that exhibit a different phenotype than the parent strain of Chlorella vulgaris are identified as the modified strain of Chlorella vulgaris. Optionally, the phenotype is at least one of: a colour, a smell, a taste, a cell wall integrity, a chitin content, a shape of the cell, growth rate, and enzyme activity, and so forth. Optionally, modified strains of Chlorella vulgaris are further selected based on one or more additional desirable phenotypes, such as growth performance and/or biochemical composition. Optionally, the strains may be selected based on texture. The chitin deficient strains of the invention may for example have the additional advantage of improved performance in food compositions.

Preferably, a reduced chitin phenotype is used as the primary method to screen for the modified strain of Chlorella vulgaris from the parent strain of Chlorella vulgaris. Specifically, the reduced chitin phenotype is defined as a permanent reduction in the assayable quantity of glucosamine, or the polymer thereof (i.e. chitin), comprising the rigid cell wall component, of the modified strain of Chlorella vulgaris as compared to the phenotype of the parent strain of Chlorella vulgaris. The reduction in the rigid cell wall component is identified by the reduced overall chitin content in the cell wall of the modified strain of Chlorella vulgaris. In such an instance, the cells of the mutated strain of Chlorella vulgaris are identified by a number of methods including calcofluor white assay and glucosamine assay. Optionally, the reduced overall chitin content in the cell wall of the modified strain of Chlorella vulgaris may be identified by mutations in the chitin synthase enzyme (EC:2.4.1.16), or mutations in genes responsible for the expression and/or regulation of the chitin synthase enzyme. Interestingly, two homologs of the chitin synthase enzyme have been identified in the genome sequence of the modified strains of Chlorella vulgaris species (Chlorella vulgaris 4TC3/16), as shown in Table 1 below. Furthermore, transcriptome analysis of a parent strain of a Chlorella vulgaris (4TC3/16) confirms expression of a putative chitin synthase gene as well as a chitin-degrading enzyme in the genome sequence of said strain of Chlorella vulgaris (4TC3/16), indicating, thereby, a natural turnover of chitin in the parent strain of Chlorella vulgaris (4TC3/16).

TABLE 1
Two homologs of chitin synthase (EC 2.4.1.16) identified in
the genome sequence of Chlorella vulgaris strain 4TC3/16

| | KEGG | | | | |
|---|---|---|---|---|---|
| Gene ID | number | Threshold | Score | E-Value | Definition |
| g7252.t1 | K00698 | 149.33 | 345.6 | 3.00E−103 | chitin synthase |
| | | | | | [EC: 2.4.1.16] |
| g7156.t1 | K00698 | 149.33 | 371.7 | 3.70E−111 | chitin synthase |
| | | | | | [EC: 2.4.1.16] |

Optionally, the identification of the modified strain of Chlorella vulgaris comprises calcofluor white staining of the cells and sorting of the cells with flow cytometry. Calcofluor white staining along with flow cytometry is a well-known technique for rapid detection of the cell wall of various organisms, such as yeast and fungi. Calcofluor white (CFW) is a fluorescent blue dye or stain that binds to β-(1-3)- and β-(1-4) polysaccharides, such as cellulose and chitin, in the cell wall. Moreover, CFW stained samples can be analysed using epifluorescence microscopy or flow cytometry for diagnosing, identifying, and counting the cells containing varying levels of chitin in cell walls. CFW has an absorption spectrum ranging from 300 to 412 nanometres (nm) with a peak at 347 nm. The CFW dye fluoresces when exposed to ultraviolet light, violet light or blue-violet light.

Flow cytometry (FCM) is a technique for detecting and measuring physical and chemical characteristics of a sample containing cells or particles. The fluorescence intensity of cells or particles stained with calcofluor white is correlated with the chitin or cellulose content of the cells or particles. The sample containing cells or particles are often labelled with fluorescent markers for analysing cells and components. Flow cytometry is based upon analysis of the relative signal strength of autofluorescence of a sample or fluorescent marker bound to a sample containing cells or particles. Optionally, flow cytometry serves as an enrichment step of physically sorting (namely, separating and isolating) chitin-reduced cells away from cells with a parental phenotype and thereby purifying cells of interest based on their specific optical properties, referred to as fluorescence-activated cell sorting or cell sorting by flow cytometry. Optionally, such isolated cells with reduced chitin content are expanded by cultivation and re-sorted through one or more additional rounds of flow cytometry to confirm the stability of the reduced chitin content phenotype or isolate a secondary mutant phenotype, for example a chlorophyll-deficient phenotype, or a colour phenotype, according to the fluorescence parameters chosen. They can then be further expanded in liquid culture or plated onto agar plus glucose plates for scoring of colours with respect to other mutations. As a control to calibrate the flow cytometry, cells of the parent strain of Chlorella vulgaris are extracted and used to calibrate the sorter as a chitin-replete control.

The method 100 comprises recovering the mutant strains of Chlorella vulgaris on a solid agar plate. Recovering the mutant strains of Chlorella vulgaris on the solid agar plate ensures isolation of only the viable cells for use in later steps of isolation of modified strains of Chlorella vulgaris. Preferably, the mutant strains are sub-cultured several times on the solid agar plates to ensure they are free from a potential contamination from bacteria or fungi. A colorimetric assay for glucosamine in a fractionated cell wall extract can be used to determine the chitin content of the modified strains in a quantitative manner. This assay comprises a cell wall preparation step (Kapaun and Reisser, 1995; DOI 10.1007/BF00191563) and the following colorimetric method used to determine glucosamine concentration (after Katano et al., 2016; DOI 10.2116/analsci.29.1021). Briefly, glucosamine reduces Mo(VI) species to form a mixed-valence molybdosilicate anion, turning the solution from yellow to blue. Using the corresponding absorbance at 750 nm a calibration curve can be generated for a chitin standard, which may be either N-acetylglucosamine (hydrolysed) or glucosamine by plotting absorbance of a series of standard solutions at 750 nm (y-axis) versus their concentration (mg/ml) (x-axis).

The quantification results from the colorimetric assay method are consistent with the qualitative results obtained from the confocal imaging following staining with calcofluor white, in confirming reduction in the overall chitin content of the modified strains of Chlorella vulgaris (namely, p5-39, p5-59, p5-94 and p4-102 with 47.48%, 52.30%, 39.70% and 37.40% reduction in the overall chitin content, respectively) as compared to the parent strain of the Chlorella vulgaris (4TC3/16), as provided in Table 2 below. All strains were analysed in biological triplicate from algal cultures harvested during exponential growth phase, and their corresponding chitin content was calculated using hydrolysed N-acetylglucosamine as the standard. It will be appreciated that assayable chitin content of both the modified and parent algal strains may naturally vary during the growth phase as a consequence of their metabolic state, in addition to other factors such as culture conditions, media composition and so forth. Notwithstanding, the modified strains of Chlorella vulgaris exhibit a quantifiable phenotype characterized by a reduced assayable chitin content, which corresponds with reduced calcofluor white fluorescence compared to the parent strain when grown under the same conditions.

TABLE 2
Overall chitin content in different strains of Chlorella vulgaris

| | Chitin | | Percentage reduction in |
|---|---|---|---|
| Strain | content | SEM chitin content | chitin content relative to |
| ID | g/g/DCW | (g/g/DCW) | the wild-type 4TC3/16 |
| 4TC3/16 | 0.00494 | 0.00012 | NA |
| p5-39 | 0.00259 | 0.00023 | 47.48 |
| p5-59 | 0.00236 | 0.00009 | 52.30 |
| p5-94 | 0.00298 | 0.00028 | 39.70 |
| p4-102 | 0.00309 | 0.00020 | 37.40 |

Optionally, the method 100 further comprises selecting healthy cells of the modified strain of Chlorella vulgaris. Optionally, the method 100 comprises filtering out unhealthy cells of the modified strain of Chlorella vulgaris, preferably by cultivation under non-permissive or stressful conditions. It will be appreciated that during mutagenesis of the parent strain of Chlorella vulgaris, cells of the modified strain of Chlorella vulgaris may acquire mutations at multiple sites within the genome, including a mutation or mutations that are causative for the desired phenotype. However, some mutated cells (strains) of Chlorella vulgaris may additionally acquire deleterious mutations as a consequence of exposure to the mutagenic agent, resulting in one or more undesired mutations, for instance in essential genes. In such an instance, it is essential to filter out these unhealthy cells of the modified strain of Chlorella vulgaris associated with the deleterious mutations, to ensure selection of only those cells which are robust and able to grow well under desired cultivation conditions. This can be achieved by cultivation of the mutated strains during the period immediately following exposure to the chemical or physical mutagen under stressed or less permissive (or non-permissive) conditions, for instance at the limit of, or slightly above the normal upper temperature for cultivation and in the absence of light but in the presence of glucose. Optionally, mutated strains are cultivated under photoautotrophic conditions, more optionally, mutated strains are cultivated under mixotrophic conditions. Only robust strains are able to proliferate under stressful conditions. This approach enriches for strains that are not compromised in their general growth characteristics. Furthermore, after cultivation, the desired phenotypes related to reduced chitin content can be scored. In such an example, the desired phenotype of the modified strain of Chlorella vulgaris is a reduced overall chitin content. Undesired phenotypes, including chitin content at levels associated with the parent strain or the wild-type strains of Chlorella vulgaris, are not selected. In other words, they are filtered out. Optionally, cells of modified strain of Chlorella vulgaris that exhibit the desired phenotype across a series of generations are selected as healthy cells. More optionally, the mutated strain of Chlorella vulgaris is cultivated at a temperature that is slightly higher than an ideal temperature (such as, above 28° C. for cultivation of the microalgal strain, to select only healthy cells of the modified strain of Chlorella vulgaris.

It will be appreciated that competition between mutated cells comprising the mutant library is a factor influencing the overall genetic diversity of the mutant library, while it is being incubated under permissive growth conditions.

Incubating the library for a number of generations following mutagenesis is a useful strategy for removing viable, but undesirable genetic mutations which adversely affect overall cell performance, or “fitness”. Incubation of algal cells immediately following mutagenesis enables the cells to recover prior to growth and assessment of chitin-deficient variants under the desired growth mode; heterotrophic, mixotrophic or autotrophic.

Optionally, the method 100 further comprises repeating steps of: performing mutagenesis of the parent strain of Chlorella vulgaris, cultivating the mutated strain of Chlorella vulgaris: at a specific temperature, for a predefined period of time, and, optionally in a mixotrophic growth mode in the presence of an organic carbon source and low light or, optionally in a heterotrophic growth mode without light and in the presence of an organic carbon source or, further optionally in the light using phototrophic growth mode; and isolating chitin-deficient mutants of the parent strain of Chlorella vulgaris. The said repetition of mutagenesis, cultivation and isolation steps enable selecting healthy cells of the modified strains of Chlorella vulgaris based on desired phenotypes (or traits) such as reduced chitin content.

The modified strain of Chlorella vulgaris has a chitin content of less than 4.8 mg/g dry cell weight. Optionally, the modified strain of Chlorella vulgaris has a chitin content of in a range of 0.001 to 4.8 milligram per gram of dry cell weight (mg/g DCW). For example, the chitin content of the modified strains of Chlorella vulgaris may be in a range of 0.001, 0.003, 0.006, 0.009, 0.012, 0.015, 0.018, 0.021, 0.024, 0.027, 0.03, 0.04, 0.05, 0.06, 0.07, 0.08, 0.09, 0.10, 0.50, 1.00 mg/g DCW up to 1.01, 1.50, 2.00, 2.50, 3.00, 3.50, 4.00 or 4.50, 4.60, 4.70 or 4.80 mg/g DCW. The modified strains of Chlorella vulgaris, namely p4-102, p5-94, p5-39 and p5-59, grown under heterotrophic conditions using an organic carbon energy source contained a chitin content of 0.00309 g/g (or 3.09 mg/g) DCW, 0.00298 g/g (or 2.98 mg/g) DCW, 0.00259 g/g (or 2.59 mg/g) DCW and 0.00236 g/g (or 2.36 mg/g) DCW, respectively as compared to a chitin content of 0.00494 g/g (or 4.94 mg/g) DCW in the parent (or wild-type) strain of Chlorella vulgaris 4TC3/16, as shown in Table 2 above.

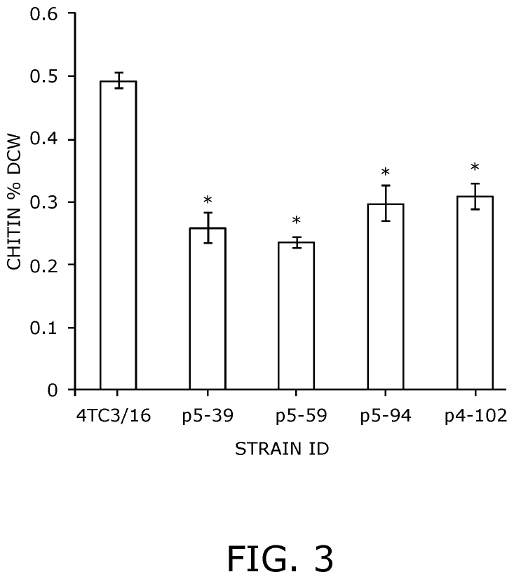

Optionally, the modified strain of Chlorella vulgaris has a greater than 10% reduction in chitin content compared with the chitin content of the parent strain of Chlorella vulgaris. A greater than 10% reduction in chitin content of the modified strain of Chlorella vulgaris compared to the chitin content of the patent strain of Chlorella vulgaris is associated with an improved PDCAAS score. The modified strains of Chlorella vulgaris, namely p4-102, p5-94, p5-39 and p5-59, grown under heterotrophic conditions using glucose as an organic carbon energy source contain a chitin content of 0.31% DCW, 0.3% DCW, 0.26% DCW and 0.24% DCW, respectively as compared to a chitin content of 0.49% DCW in the parent (or wild-type) strain of Chlorella vulgaris 4TC3/16, as shown in FIG. 3. Specifically, chitin content in the modified strains of Chlorella vulgaris (namely, p4-102, p5-94, p5-39 and p5-59) records approximately 37.40%, 39.70%, 47.48% and 52.30% reduction in the chitin content as compared to the parent (or wild-type) strain of Chlorella vulgaris 4TC3/16, respectively.

Optionally, the modified strain of Chlorella vulgaris has one or more additional desirable phenotypes. The one or more additional desirable phenotypes being positively associated with economical production, compositional functionality or organoleptic properties of the strain. Optionally, the one or more additional desired phenotypes is selected from one of: a colour, a pigment content, a protein content, a smell, a taste, a texture, a structural integrity, a biochemical composition and improved tolerance to process conditions (growth rate in permissive as well as non-permissive conditions). Optionally, the phenotype is a scorable phenotype, wherein such phenotypes may be identifiable by various methods for such identification known to a person skilled in the art. In an example, apart from the desirable chitin-reduced cell wall phenotype, the modified strains of Chlorella vulgaris may be isolated with other desirable properties, such as for example reduced chlorophyll content as assayed, for example, by HPLC or other suitable method, in the modified strains of Chlorella vulgaris.

Optionally, the colour of the modified strain of Chlorella vulgaris is one of: white, cream, pale yellow, yellow, pale green, golden, orange, brown, pink, red or lime colour, with the colour also being associated strongly with smell and taste. Typically, the colour may be determined by visual inspection of the strains, however, other analytical methods may also be used to determine and measure the colour of the modified strains of Chlorella vulgaris.

Alternatively, the modified strain of Chlorella vulgaris is incapable of producing, or has substantially reduced ability to produce chlorophyll pigments (chlorophyll a and/or chlorophyll b), however possesses a variable, but genetically-determined ability to produce other pigments, such as for example lutein, xanthophylls other carotenoids and tetrapyrroles. As a result, the colour of such Chlorella vulgaris strains is one of: pink, red, red-brown, brown or yellow-brown colour. Moreover, the content of chlorophyll a, chlorophyll b and/or lutein and/or other pigments in the modified strain of Chlorella vulgaris can be determined using analytical methods known to the skilled person, for example chromatographic or spectrophotometric techniques.

Optionally, the modified strain of Chlorella vulgaris has a chlorophyll content in a range of 0.001 to 0.50 mg/g dry cell weight, preferably 0.25 to 0.50 mg/g dry cell weight, 0.10 to 0.25 mg/g dry cell weight, or 0.001 to 0.1 mg/g dry cell weight when grown under heterotrophic growth conditions. For example, the chlorophyll content of the modified strain of Chlorella vulgaris may be 0.001, 0.002, 0.003, 0.004, 0.005, 0.01, 0.015, 0.02, 0.025, 0.03, 0.035, 0.04, 0.045, 0.05, 0.1, 0.15, 0.2, 0.25, 0.3, 0.35, 0.4 or 0.45 mg/g DCW up to 0.002, 0.003, 0.004, 0.005, 0.01, 0.015, 0.02, 0.025, 0.03, 0.035, 0.04, 0.045, 0.05, 0.1, 0.15, 0.2, 0.25, 0.3, 0.35, 0.4, 0.45 or 0.50 mg/g DCW, preferably 0.25, 0.3, 0.35, 0.4 or 0.45 mg/g DCW up to 0.3, 0.35, 0.4, 0.45 or 0.50 mg/g DCW, 0.1, 0.15 or 0.2 mg/g DCW up to 0.15, 0.2 or 0.25 mg/g DCW, or 0.001, 0.002, 0.003, 0.004 or 0.005 mg/g DCW up to 0.001, 0.002, 0.003, 0.004, 0.005 or 0.1 mg/g DCW. Notably, lower chlorophyll content of the modified strain of Chlorella vulgaris renders the modified strain of Chlorella vulgaris more commercially acceptable. For example, a modified strain of Chlorella vulgaris with chlorophyll content of 0.001 mg/g DCW will be more commercially acceptable in industries that require no colour in their final manufactured products, as compared to the modified strain of Chlorella vulgaris with chlorophyll content of 0.10 mg/g DCW. Beneficially, the modified strain of Chlorella vulgaris having the reduced chlorophyll content is a potential ingredient in various food and personal care applications. Furthermore, the reduced chlorophyll content of the modified strain of Chlorella vulgaris is also associated with reduction in the unpleasant colour, smell and taste (organoleptics) associated with the wild-type strain of Chlorella vulgaris, when used in the food and personal care applications. Additionally, beneficially, the modified strain of Chlorella vulgaris having the reduced chlorophyll content can be incorporated at a higher percentage as an ingredient in food compositions, compared with the wild-type, as a result of such improvements in the organoleptic properties.

Optionally, the modified strain of Chlorella vulgaris has a lutein content lower than the lutein content of the parent strain of Chlorella vulgaris when grown heterotrophically. Optionally, the modified strain of Chlorella vulgaris has a lutein content lower than the lutein content of the parent strain of Chlorella vulgaris, normally in a range of 3 to 10 mg/g DCW when grown heterotrophically. For example, the lutein content of the parent strain of Chlorella vulgaris may be 3, 3.5, 4, 4.5, 5, 5.5, 6, 6.5, 7, 7.5, 8, 8.5, 9 or 9.5 mg/g DCW up to 3.5, 4, 4.5, 5, 5.5, 6, 6.5, 7, 7.5, 8, 8.5, 9, 9.5 or 10 mg/g DCW. The average normal amount of lutein in the parent strain of Chlorella vulgaris is 5 mg/g DCW. Optionally, the modified strain of Chlorella vulgaris has a lutein content below 9 mg/g DCW, more optionally below 8 mg/g DCW, yet more optionally below 7 mg/g DCW, yet more optionally still below 6 mg/g DCW, yet more optionally still below 5 mg/g DCW, yet more optionally below 4 mg/g DCW, yet more optionally still below 3 mg/g DCW, yet more optionally still below 2 mg/g DCW, yet more optionally still below 1 mg/g DCW, and yet more optionally up to 0.1 mg/g DCW of the lutein content of a parent strain of Chlorella vulgaris. Notably, lower lutein content of the modified strain of Chlorella vulgaris renders the modified strain of Chlorella vulgaris more commercially acceptable. For example, a modified strain of Chlorella vulgaris with a lutein content of 0.01 mg/g DCW will be more commercially acceptable for certain applications, as compared to the modified strain of Chlorella vulgaris with lutein content of 1 mg/g DCW. The reduced lutein content in the modified strain of Chlorella vulgaris results in reduced orange-red pigmentation in the modified strain of Chlorella vulgaris as compared to the parent strain of Chlorella vulgaris. In concert with the reduced chlorophyll content in certain strains this can result in a genetically determined colour form of Chlorella vulgaris with lime, pale green or even a white appearance when grown under the same conditions as the parent strain. Beneficially, the modified strain of Chlorella vulgaris having the reduced lutein content is a potential ingredient in various food and personal care applications.

Optionally, the modified strain of Chlorella vulgaris has a protein digestibility-corrected amino acid score (PDCAAS) in a range of 0.75 to 1.0. Preferably, the protein digestibility-corrected amino acid score (PDCAAS) is in a range of 0.75 to 0.831, and more preferably in a range of 0.75 to 0.79. The typical PDCAAS of the modified strain of Chlorella vulgaris may be for example in a range from 0.75, 0.80, 0.85, 0.90 or 0.95 up to 0.80, 0.85, 0.90, 0.95 or 1.0, preferably in a range from 0.75, 0.76, 0.77, 0.78, 0.79, 0.80, 0.81 or 0.82 up to 0.76, 0.77, 0.78, 0.79, 0.80, 0.81, 0.82 or 0.831, and more preferably in a range from 0.75, 0.76, 0.77 or 0.78 up to 0.76, 0.77, 0.78 or 0.79. Therefore, a PDCAAS value in a range of 0.75 to 1.0, preferably in a range of 0.75 to 0.831, and more preferably in a range of 0.75 to 0.79 means that, by weight, the said protein source will provide essential amino acids for human needs in a range of 75 to 100%, preferably in a range of 75 to 83.1%, and more preferably in a range of 75 to 79%. The modified strain of Chlorella vulgaris, namely p5-59, has a PDCAAS score of 0.79 (79%) as compared to the PDCAAS score of 0.75 (75%) of the parent (wild-type) strain of Chlorella vulgaris, namely 4TC3/16.

Beneficially, the present disclosure uses the Protein Digestibility Assay Kit™ (K-PDCAAS™) (using the Animal-Safe Accurate Protein Quality Score (ASAP-Quality Score Method) developed under U.S. Pat. No. 9,738,920 by Medallion Labs), manufactured by Megazyme, Ireland to test the protein quality in an ethical manner, such as by avoiding traditional in vivo rat assay (animal-based studies) used in typical PDCAAS assays. This means the Chlorella vulgaris biomass of the present disclosure remains suitable for a vegan label.

Optionally, the modified strain of Chlorella vulgaris maintains a minimum protein content of 20%, or optionally, 25%, or optionally 30%, or optionally 35% protein, or optionally 40% w/w, or optionally 45% w/w, and still more optionally 50% w/w. For example, the protein content of the modified strain of Chlorella vulgaris may be from 20%, 25%, 30%, 35%, 40% or 45% up to 30%, 35%, 40%, 45% or 50% w/w. It will be appreciated that it is possible that some strains may be used to produce biomass with more than 50% w/w of protein content.

Optionally, the modified strain of Chlorella vulgaris is selected based on a desirable protein content, wherein the desirable protein content is based upon a relative signal obtained on cell sorting by flow cytometry.

Furthermore, the modified strain of Chlorella vulgaris of the present disclosure is genetically stable. In an example, the genetically stable strains of Chlorella vulgaris are genetically determined to have a reduced chitin content, and optionally one of: white, cream, pale yellow, yellow, pale green, golden, orange, brown, pink, red or lime colour determined by lack of or reduced production of chlorophyll and/or other pigments in the modified strain of Chlorella vulgaris, and in some instance production of increased amounts of intermediate pigments resulting from the underlying genetic changes. Furthermore, the genetically stable strain of Chlorella vulgaris does not revert to characteristics associated with the parent strains of Chlorella vulgaris even under alternate growth conditions and over time (such as, over multiple hundreds of generations of cultivation).

The parent strains of Chlorella vulgaris are haploid. A haploid parent strain prevents the modified strains thereof from reverting back from a desired genotype to the genotype commonly associated with the parent strain of Chlorella vulgaris over successive generations of cultivation, beneficially exhibiting relative stability of the desired phenotype in such strains. The modified strain of Chlorella vulgaris is genetically stable with respect to the observed reduced cell wall rigidity phenotype. Notably, the quantitative analysis, including calcofluor white staining and flow cytometry, or optionally, qualitatively, calcofluor white staining and confocal microscopy, of strains of Chlorella vulgaris maintained both on agar and in liquid culture is sufficient to conclude that the phenotype, such as reduced chitin, is genetically stable in the modified strain of Chlorella vulgaris. Further, the stability of genetic mutations can also be confirmed by direct genetic sequencing.

Furthermore, the modified strain of Chlorella vulgaris is genetically stable and can be grown under heterotrophic growth conditions, and over time with the desired phenotype including reduced chitin content, improved colour, smell and taste parameters that are more suitable for human consumption and potentially more suitable for incorporation into certain animal feeds that may be limited by chlorophyll incorporation threshold. Consequently, such modified strains of Chlorella vulgaris may find potential applications as whole food or as an ingredient in human (and animal) foods, nutraceutical preparations, cosmetic formulations, medicines, personal care, and so on, owing to increased market uptake/acceptance, preferable organoleptic properties which optionally, additionally, enable a higher incorporation rate in food products and improved digestibility as modelled by PDCAAS.

Another embodiment of the present disclosure provides a composition comprising an algae biomass derived from the modified strain of Chlorella vulgaris. The algal biomass is obtained from the modified strain of Chlorella vulgaris having a chitin content lower than a chitin content of a parent strain of Chlorella vulgaris grown under the same conditions, such as heterotrophic, mixotrophic or autotrophic growth conditions. Optionally, the algal biomass has a chitin content of less than 4.8 mg/g dry cell weight, preferably the chitin content is in a range of 0.001 to 4.8 mg/g dry cell weight. Furthermore, such an algae biomass can be obtained by performing the method 100 of producing the modified strain of Chlorella vulgaris (as explained in detail hereinabove). Optionally, the algae biomass derived from the modified strain of Chlorella vulgaris has a chitin content of less than 4.8 mg/g DCW, preferably 0.001 to 4.8 mg/g DCW. Beneficially, the chitin-deficient phenotype in the algal biomass results in high digestibility of said algal biomass by human or animal consumer, assayed by PDCAAS.

Optionally, the modified strain of Chlorella vulgaris is improved with regard to the energy required to process the resulting biomass for any application. The reduced chitin content removes the need for or lowers the energy intensity in any downstream processing of the biomass to aid digestion and/or nutrient availability (e.g. by physical, chemical or enzymatic means), thereby reducing the cost, technical- or energy requirement of downstream processing, for example cracking, milling, or extruding of the Chlorella vulgaris biomass The modified strain of Chlorella vulgaris finds application in various consumer products, including but not limited to food, beverage, cosmetic and personal care, by reducing the need for downstream processing of the Chlorella vulgaris biomass to aid digestion and/or nutrient availability. It is understood by a person skilled in the art that a reduced energy intensity in relation to any normally required downstream processing steps would result in a reduced carbon footprint of the overall process. It is further understood that the same reduced chitin content, beneficially enables improvements in the overall economic return of investment related to biofuels production from the said organism.

Optionally, the composition is a food or food ingredient. Furthermore, the algal biomass of the present disclosure does not impart the dark green colour, unpleasant smell or taste associated with the wild-type strains of Chlorella vulgaris, to the processed food product. Beneficially, the modified strain of Chlorella vulgaris of the present disclosure is classed as a non-genetically modified (non-GM), i.e. is not a genetically modified organism, and currently classed as a not genetically modified food source. Additionally, the modified strain of Chlorella vulgaris is nutritious (comprising high protein and fibre content in addition to a range of B and E vitamins), gluten free and animal-free (vegetarian and/or vegan) and has been described as a super food and super ingredient.

Optionally, the composition comprising algae biomass derived from the aforementioned modified strain of Chlorella vulgaris or obtained by performing the aforementioned method 100, is employed as an ingredient in at least one of: human foods, human nutraceutical preparations or formulations, animal feeds, drug compositions, cosmetics, personal care compositions, personal care devices. It will be appreciated that the reduced chitin content enables enhanced nutrient availability and removes the need for downstream processing of the biomass to aid digestion and/or nutrient availability. Moreover, the reduced chlorophyll content and optionally, the reduced content of other pigments, including but not limited to lutein, provides the modified strain of Chlorella vulgaris one or more improved organoleptic properties, such as appealing appearance, smell and/or taste. Consequently, a reduced chitin content and such one or more parameters of the modified strain of Chlorella vulgaris enable usage in an expanded range of products, and beneficially, (particularly with respect to the content of desirable macro and micronutrients aforementioned), at higher incorporation rates in products used or consumed by humans and/or animals. The food for humans can include but is not limited to bakery products, pastas, cereals, cereal bars, confections, sauces, soups, dairy substitutes, frozen desserts, ice creams, yoghurts, smoothies, creams, spreads, salad dressings, mayonnaises, food garnishing and seasoning, candies, gums, jellies, vape liquid and so forth. Moreover, the algae biomass obtained from the modified strain of Chlorella vulgaris can be used as an ingredient for example in nutraceutical preparations and formulation for humans, pharmaceutical compositions, cosmetics, personal care compositions, personal care devices and so forth. The nutraceutical preparations and formulation comprise, for example, nutritional supplements, hormone tablets, digestive capsules, tablets, powders, oils and the like. The cosmetic formulations may employ use of the algae biomass or specific extracts derived therefrom, for example, in lipsticks, powders, creams, exfoliants, facial packs, and so forth. The personal care compositions and personal care devices comprise toothpastes, mouthwash, hand-wash, body-wash, body soaps, shampoos, oils, sun-creams, after-sun creams, sunblock and so forth. The pharmaceutical compositions include any type of compositions known to the skilled person for the delivery of medicaments, including bioactives, vaccines and delivery vehicles for other recombinant proteins and enzymes.

Yet another embodiment of the present disclosure provides a method of using the aforementioned composition as an ingredient in at least one of: human foods, human nutraceutical preparations or formulations, animal feeds, pharmaceutical compositions including vaccines, cosmetics, personal care compositions, personal care devices or textiles, dyes, inks or for the production of fuels. The method of use comprises using the algae biomass ingredient comprising the modified strain of Chlorella vulgaris as any one of: a dried powder, dried flakes, a frozen paste, an extract (aqueous or polysaccharide extract), solutions, suspensions, solution preconcentrates, emulsions, emulsion pre-concentrates, a concoction, tablets, pills, pellets, capsules, caplet, concentrates, granules, and so forth. Furthermore, a dried, fresh, or frozen part of the modified strain of Chlorella vulgaris, oil derived from the modified strain of Chlorella vulgaris, a homogenate, whole cell, lysed cell and so forth can be used in preparation of human foods, human nutraceutical preparations or formulations, animal feeds, pharmaceutical compositions, cosmetics, personal care compositions, personal care devices and fuels. Moreover, there is provided a method of using the algae of the invention as an ingredient in various food, personal care, medicinal and nutritional applications comprising combining the food or food ingredient with one or more additional edible or suitable ingredients, such as milk, oil, cream, water, spices, herbs, minerals, proteins, one or more chemical compounds, preservatives, aromatic juices, and the like, to obtain the desired human foods, human nutraceutical preparations or formulations, animal feeds, pharmaceutical compositions, cosmetics, personal care compositions or personal care devices. Moreover, the modified strain of Chlorella vulgaris can be used to prepare compositions in any way known to the skilled person. Beneficially, the modified strain of Chlorella vulgaris requires fewer passes through a microfluidizer (such as a Microfluidics MP110 Microfluidizer at 30 k psi) to achieve the same percentage of cell lysis relative to the parent strain of Chlorella vulgaris (Table 3) Improved cell lysis corresponds to an increase in the assayable levels of free protein in the homogenate, for example.

TABLE 3
Percentage of cell lysis following up to 3 passes in a Microfluidics
MP110 Microfluidizer at 30k psi for two chlorophyll-deficient parental
strains of Chlorella vulgaris (WC03 and LC01) and a chitin-deficient,
chlorophyll-deficient strain of Chlorella vulgaris (CM/YC01).

| | Pass through | Percentage of cells |
|---|---|---|
| Strain | microfluidizer | lysed (SEM) |
| WC03 | 1 | 63 (4.0) |
| | 2 | 84 (2.5) |
| | 3 | 95 (2.4) |
| LC01 | 1 | 61 (1.9) |
| | 2 | 79 (0.3) |
| | 3 | 89 (6.7) |
| CM/YC01 | 1 | 82 (0.5) |
| | 2 | 92 (2.4) |
| | 3 | 97 (1.4) |

Yet another embodiment of the present disclosure provides a microalgae flour comprising a homogenate of microalgae biomass derived from the modified strain of Chlorella vulgaris of the invention or obtained by performing the method 100 of producing the modified strain of Chlorella vulgaris. The microalgae flour comprises the homogenate of microalgae biomass derived from the modified strain of Chlorella vulgaris. Furthermore, the microalgae flour is obtained by performing the method 100 of producing the modified strain of Chlorella vulgaris (as described in detail hereinabove).

Beneficially, the reduced chitin content of the modified strain of Chlorella vulgaris improves a genetic transformation efficiency of the modified strain of Chlorella vulgaris. The genetic transformation of the modified strain of Chlorella vulgaris with heterologous DNA may be achieved using conventional techniques, such as for example nanoparticle-based gene gun (“biolistics”) and electroporation. A lower chitin content of the cell wall provides a reduced physical barrier to recombinant or heterologous DNA, thereby directly enabling enhanced biotechnology application of modified strain of Chlorella vulgaris, such as genetic engineering. In such case, the modified strain of Chlorella vulgaris is more susceptible to genetic transformation using gold nanoparticle biolistics or electroporation methods as compared to the parent strain of Chlorella vulgaris.

Additionally, beneficially the reduced rigid cell wall of the modified strain of Chlorella vulgaris may enhance an overall cell division rate by reducing the time-dependent deposition and dissolution of the cell wall during each cell division. The utilization of the carbon sink of the cell wall and increase in the overall cell division rate in the modified strain of Chlorella vulgaris could be positively associated with the industrial application of such modified strains. Furthermore, reduction of the carbon sink associated with the cell wall could increase the relative desirable percentage of other cellular components including but not limited to total protein.

Optionally, the modified strain of Chlorella vulgaris is genetically stable and exhibits improved permeability to exogenous DNA, RNA, protein, polypeptides or complexes derived therefrom as compared to its parent strain. The less rigid cell wall opens avenues for the use of the modified strains of the microalgae, Chlorella vulgaris, for various biotechnology applications, such as recombinant DNA technology, and so on. The genetic transformation of the modified strain of Chlorella vulgaris with heterologous DNA may be achieved using conventional techniques, such as for example nanoparticle-based gene gun (“biolistics”) and electroporation. A lower chitin content of the cell wall provides a reduced physical barrier to recombinant or heterologous DNA, thereby directly enabling enhanced biotechnology application of modified strain of Chlorella vulgaris, such as genetic engineering. In such case, the modified strain of Chlorella vulgaris is more susceptible to genetic transformation using gold nanoparticle biolistics or electroporation methods as compared to the parent strain of Chlorella vulgaris.

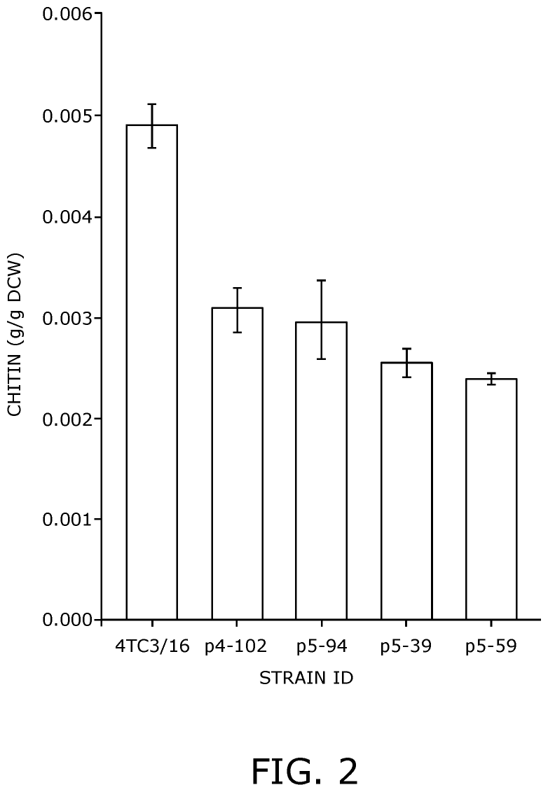

FIG. 2 shows chitin content in modified strains of Chlorella vulgaris represented as gram/gram dry cell weight (g/g DCW) as compared to chitin content in parent (wild-type) strain of Chlorella vulgaris cultivated under the same conditions. The relative amount of chitin content (g/g DCW) in the modified strains of Chlorella vulgaris is represented in relation to the chitin content in the parent (wild-type) strain of Chlorella vulgaris 4TC3/16 grown under heterotrophic conditions using an organic carbon energy source. As shown, chitin content (g/g DCW) of the parent (or wild-type) strain of Chlorella vulgaris 4TC3/16 and various chitin-deficient mutants of the parent strain 4TC3/16, namely p4-102, p5-94, p5-39 and p5-59, is provided. Error bars are the standard deviation of three technical repeats of a glucosamine-detecting chitin hydrolysis colorimetric assay for each strain is provided corresponding to the respective strain of Chlorella vulgaris. 4TC3/16 strain has a chitin content of 0.00494 g/g dry cell weight (DCW), p4-102 strain has a chitin content of 0.00309 g/g DCW, p5-94 strain has a chitin content of 0.00298 g/g DCW, p5-39 strain has a chitin content of 0.00259 g/g DCW and p5-59 strain has a chitin content of 0.00236 g/g DCW. Notably, 4TC3/16 strain exhibits the presence of the highest measured chitin content. Modified strains of Chlorella vulgaris, namely p4-102, p5-94, p5-39 and p5-59, exhibit a significant and reproducible reduction in chitin content.

FIG. 3 shows chitin content in modified strains of Chlorella vulgaris represented as a percentage dry cell weight (% DCW) compared to chitin content in parent (wild-type) strain of Chlorella vulgaris cultivated under the same conditions, in accordance with an embodiment of the present disclosure. The relative amount of chitin content (% DCW) in the modified strains of Chlorella vulgaris is represented in relation to the chitin content in the parent (wild-type) strain of Chlorella vulgaris 4TC3/16 grown under heterotrophic conditions using an organic carbon energy source. As shown, a percentage of chitin content (% DCW) in the parent (or wild-type) strain of Chlorella vulgaris 4TC3/16 and various chitin-deficient mutants of the parent strain 4TC3/16, namely p5-39, p5-59, p5-94, and p4-102, is provided. Error bars are the standard deviation of multiple technical repeats of a glucosamine-detecting chitin hydrolysis colorimetric assay for each strain. As shown, 4TC3/16 strain has the highest chitin content of approximately 0.49% dry cell weight (DCW), p5-39 strain has a chitin content of 0.26% DCW, p5-59 strain has a chitin content of 0.24% DCW, p5-94 strain has a chitin content of 0.3% DCW, and p4-102 strain has a chitin content of 0.31% DCW. The chitin-deficient strain of Chlorella vulgaris (p5-59) records approximately a 50% reduction in the chitin content as compared to the parent (or wild-type) strain of Chlorella vulgaris 4TC3/16.

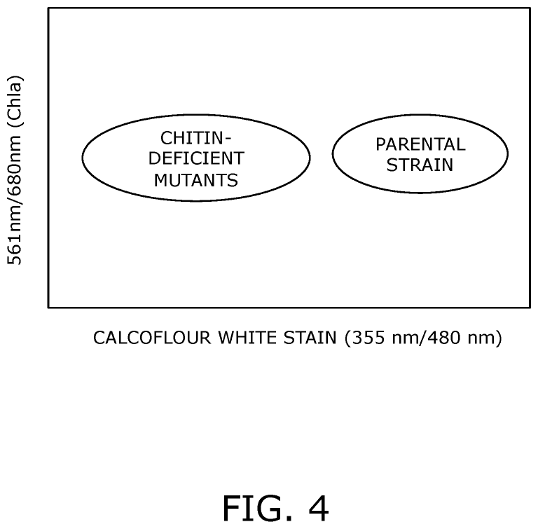

FIG. 4 shows a schematic illustration of a flow cytometry method to identify subpopulations of cells based on chitin content, following staining with calcofluor white. As shown, the Y-axis depicts the fluorescence intensity from a specific wavelength emission window attributed to chlorophyll a following excitation at a specific wavelength. The X-axis depicts the fluorescence intensity from a specific wavelength emission window attributed to calcofluor white following excitation at a specific wavelength. It will be appreciated from this diagram that cells of the parental strain and cells that are chitin reduced thereof may be separated by their differential fluorescence profiles using an appropriate gating strategy—represented by adjacent ovals, which define the two groups of cells.

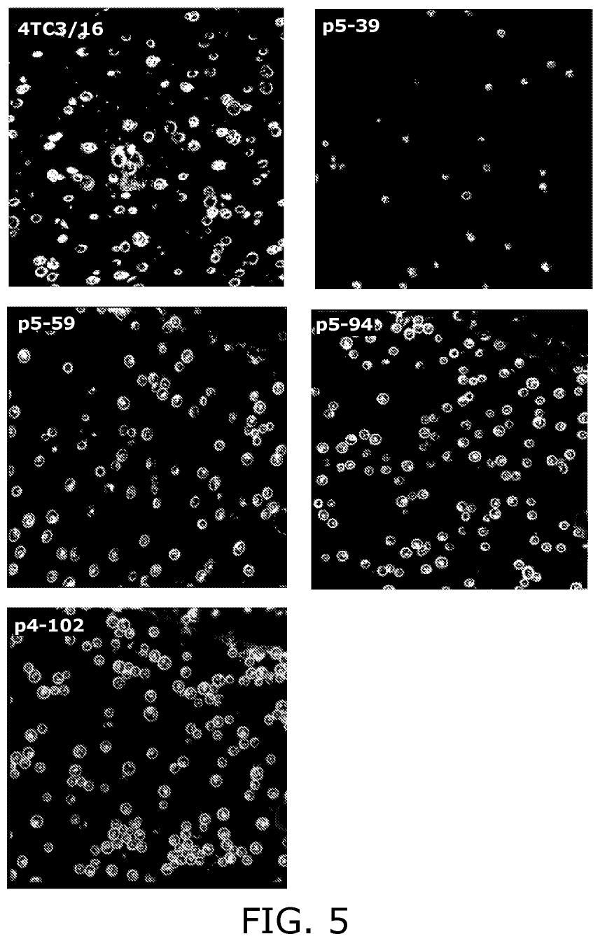

FIG. 5 shows chitin content in modified strains of Chlorella vulgaris represented as fluorescence in a confocal calcofluor white staining compared to chitin content in parent (wild-type) strain of Chlorella vulgaris cultivated under the same conditions. As shown, the bright fluorescence exhibits a high chitin content in the parent (or wild-type) strain of Chlorella vulgaris 4TC3/16, while the level of fluorescence in the various chitin-deficient mutants of the parent strain 4TC3/16, namely p5-39, p5-59, p5-94, and p4-102, is consistent with a reduced chitin content in said modified strains of Chlorella vulgaris.

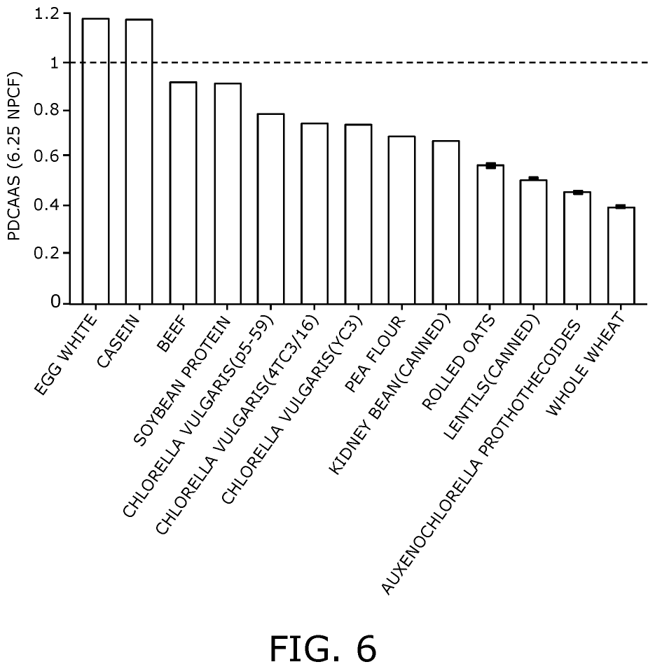

FIG. 6 shows a comparison of protein digestibility-corrected amino acid score (PDCAAS) for a range of protein sources. As shown, the protein digestibility-corrected amino acid score (PDCAAS) for a range of protein sources (data published as Protein quality evaluation: report of the Joint FAO/WHO Expert Consultation, Bethesda, Md., USA 4-8 Dec. 1989), including parent (or wild-type) strain of Chlorella vulgaris 4TC3/16, Chlorella vulgaris YC3 (a genetically stable, yellow, chlorophyll-deficient strain derived previously from Chlorella vulgaris 4TC3/16) and Chlorella vulgaris chitin-reduced variant strain p5-59 (experimental data corresponding to the present disclosure) is provided. The final PDCAAS score was calculated using the Dumas method for protein assay and a nitrogen to protein conversion factor of 6.25 to normalise the calculated score to other protein sources for comparison. Data was generated using the Protein Digestibility Assay Kit (K-PDCAAS; Megazyme, Ireland). By convention, PCDAAS scores are truncated at 1 (100%); dashed line. Chlorella vulgaris 4TC3/16 exhibits a better PDCAAS score (74.98%) than a range of other protein sources, including, but not limited to pea protein (69.52%), rolled oats (57.33%) and the microalgae Auxenochlorella protothecoides (45.89%).

As shown, chlorophyll deficiency alone does not result in an improved PDCAAS score (74.28% for YC3), compared to the wild-type parent strain. In contrast, a chitin-deficient variant of Chlorella vulgaris 4TC3/16 of the invention (namely, p5-59) exhibits an improved PDCAAS score (78.96%) compared to either wild-type or chlorophyll-deficient strain YC3 of Chlorella vulgaris 4TC3/16. This score indicates improved digestibility of biomass derived from the chitin-reduced strain taking it closer to the PDCAAS score of soybean protein (92.12%) than its parent strain.

FIG. 7 shows an illustration of chitin biosynthesis in parent (or wild-type) strain of Chlorella vulgaris 4TC3/16. Enzyme activities specific to Chlorella vulgaris 4TC3/16 (highlighted) were functionally assigned using PANTHER and eggNOG databases, and supplemented by manual checking for missing activities using BLASTP in the 4TC3/16 proteome.

The present invention is advantageous as it provides a genetically stable, modified strain of Chlorella vulgaris having a reduced chitin content. The chitin content is lower than the chitin content of a wild-type strain of Chlorella vulgaris grown under the same conditions. Additionally, the modified strain of Chlorella vulgaris may be further improved by the addition of other desirable phenotypes such as low chlorophyll content that are also determined as stable genetically changes. Furthermore, the method ensures reduction in the chitin content of the existing parent strains (or a wild-type strain of Chlorella vulgaris) while identifying those modified strains of Chlorella vulgaris where the genetic changes do not result in a negative impact on growth under commercial scale cultivation conditions. Moreover, any modified cells of the parent strain of Chlorella vulgaris that might have displayed the desired change in chitin content, but may have also acquired deleterious mutations are filtered out, thus, providing robust, commercially scalable strains of the modified strain of Chlorella vulgaris having the desired phenotype.

Furthermore, the mutagenesis conditions comprise the use of a non-lethal (minimal dosage) quantity of the mutagenic chemical, thus, enabling the derivation of the desired phenotype in the modified strain of Chlorella vulgaris while balancing against excessive accumulation of undesirable mutations that reduce cell fitness thereof. Consequently, the method affords derivation of modified strains that retain the robust growth characteristics of the parent strain, suitable for production at commercial scale. The non-genetically modified strain of Chlorella vulgaris exhibiting chitin-deficient phenotype results in a reduced rigid cell wall and aids in digestibility and nutrient availability. The modified strain of Chlorella vulgaris finds application in various consumer products, including but not limited to food, beverage, cosmetic and personal care, by reducing the need for downstream processing of the Chlorella vulgaris biomass to aid digestion and/or nutrient availability.

Additionally, beneficially, the reduced chitin content in the modified strain is a result of a stable genetic mutation that is non-genetically modified and is non-transgenic. The stable genetic mutation resulting in the overall reduced chitin content enhances the digestibility of such microalgae, Chlorella vulgaris, by humans as well as animals, and is considered safe to eat for both humans and animals both as a whole food and as an ingredient. Reduced rigidity in the cell wall as a result of substantial reduction in the chitin content improves the nutrient bioavailability to the consumers. Furthermore, the less rigid cell wall opens avenues for the use of the modified strains of the microalgae, Chlorella vulgaris, for various biotechnology applications, such as recombinant DNA technology, and so on. Furthermore, it is considered to be reasonable that an individual skilled in the art could use the same approach exemplified in this application to create similar chitin-deficient or reduced variants of other cell wall polysaccharides or proteins from other green algae species, especially those selected from within the Chlorellaceae that may have a chitin or chitosan component to their cell walls including but not limited to Chlorella sorokiniana, Parachlorella kessleri, Auxenochlorella protothecoides, Auxenochlorella pyrenoidosa, and Heterochlorella luteoviridis. It is also well-understood that, for some algal species, independently isolated wild-type strains may naturally contain different quantities of assayable chitin when compared with each other, and consequently, such variation, where it exists, is not a “reduction” of chitin content of one compared with the other according to the invention.

For example, two strains of wild-type Chlorella sorokiniana; UTEX B3016 and UTEX 1230, were cultivated under the same growth conditions, in the same growth media and their respective chitin content assayed at both exponential and stationary phase, as described herein above, using N-acetylglucosamine (hydrolysed) as the standard (Table 4) It was observed that Chlorella sorokiniana UTEX 1230 consistently exhibited a naturally lower chitin content than Chlorella sorokiniana UTEX B3016.

TABLE 4
Overall chitin content in different strains of Chlorella sorokiniana

| | | Chitin content (g/g/DCW) |
|---|---|---|
| | Growth | N-acetylglucosamine (hydrolysed) |
| Strain | phase | standard Mean (SEM) |
| UTEX B3016 | Exponential | 4.97 (0.34) |
| UTEX B3016 | Stationary | 5.31 (0.41) |
| UTEX 1230 | Exponential | 2.70 (0.33) |
| UTEX 1230 | Stationary | 2.43 (0.37) |

Modified strains of Chlorella sorokiniana, derived from strains UTEX B3016 and UTEX1230, according to the invention, have a chitin content at least 10% lower than the chitin content of the strains from which they are derived, when grown under the same conditions.

Modifications to embodiments of the invention described in the foregoing are possible without departing from the scope of the invention as defined by the accompanying claims. Expressions such as “including”, “comprising”, “incorporating”, “consisting of”, “have”, “is” used to describe and claim the present invention are intended to be construed in a non-exclusive manner, namely allowing for items, components or elements not explicitly described also to be present. Reference to the singular is also to be construed to relate to the plural. Numerals included within parentheses in the accompanying claims are intended to assist understanding of the claims and should not be construed in any way to limit subject matter claimed by these claims.

### Experimental Details

Genetically Defining Chlorella vulgaris Strains Using PCR Amplification: Chlorella vulgaris 4TC3/16 was Genetically Defined by 18S and ITS2 Sequencing as Described Above.

Cultivating Chlorella vulgaris strains under heterotrophic growth mode: Briefly, Chlorella vulgaris strains were grown in 20 millilitre (ml) of liquid medium containing glucose or acetate at a starting cell density of 2×10⁶ cells/ml. Cells were grown in the dark at 26° C. for 6 days. A 10 ml aliquot was removed and centrifuged (4500×g, 10 minutes) to collect the cells; the pellets were washed in 1 ml double-distilled (dd) H₂O and centrifuged again (4500×g, 10 minutes). The resulting biomass pellets were dried by lyophilisation in pre-weighed tubes. Once dry, the dry cell weight (DCW) was determined before carrying out the extraction.

Selecting viable cells of the modified strains of Chlorella vulgaris: Cells growing as colonies of the modified strains of Chlorella vulgaris associated with the desired phenotypes were isolated and streaked sequentially and iteratively on a solid medium to obtain axenic, isogenic strains as well as to assess the stability of the chitin-reduction phenotype under conditions more approximating a commercial cultivation scheme. The colonies were further inoculated using a liquid media. The liquid media was at least one of TAP (Tris-Acetate-Phosphate), High Salt Medium™ (HSM™) plus glucose (for example, having 1 to 3% w/v glucose). More optionally, the colonies were cultivated in dark conditions at the specific temperature of 25° C. (or between 20 and 35° C.) for 1 to 3 weeks (or between 1 to 5 weeks) and monitored over multiple successive generations for stable phenotypes. Such stable phenotypes were associated with reduced chitin content within the modified strains of Chlorella vulgaris.

Identifying and isolating modified strains of Chlorella vulgaris: The recovered mutant strains were resuspended in a phosphate-buffered saline, stained with calcofluor white (CFW) fluorescent dye and sorted by using flow cytometry according to their fluorescence shift at 380 nm and compared to the non-mutated parent strain of Chlorella vulgaris (namely, control). Briefly, a single suspension of cells was prepared, effectively stained and allowed to flow through the flow cytometer in a single flow through the light beam for sensing. The laser was used to elicit strong chitin autofluorescence from a mixture of viable cells. The dye-specific autofluorescence signals were analysed by a computed physically connected to the flow cytometer. The population of cells that exhibit strong autofluorescence is sorted away from those cells that have null or significantly reduced signal as an enrichment step to enrich for those cells within the total population that have accumulated mutations that knock down or abolish the chitin content signal. This step was applied optionally between 2-7 days post-exposure to mutagen and in liquid culture.

Calcofluor white (CFW) staining: Typically, before staining, a stock solution of CFW was prepared by adding 35 milligram (mg) of CFW to 7 millilitre (ml) of sterile distilled water, and a few drops of 10 Normal (N) sodium Hydroxide (NaOH) was added to increase the pH of said stock solution to 10 to 11 and increase solubility of CFW in sterile distilled water. A final stock solution (adjusted to 10 ml with addition of sterile distilled water) was divided into small aliquots (for example, of 150 microlitre (μl), and stored in the dark at a temperature of −20° C. until use. Alternatively, instantaneous staining with one or two drops of 0.1% CFW was used to stain a sample, such as strains of Chlorella vulgaris, for detection of chitin in the cell wall and stained for a time period of, for example, 1 minute or less before analysing.

Cell sorting using Flow Cytometry: Briefly, the sample containing cells or particles was suspended in a fluid and injected into a flow cytometer instrument. The flow rate was controlled to allow one cell at a time to flow through a laser beam (561/680 nm), where the light scattered is characteristic to cells and components thereof.

Optionally, the flow cytometry gates were set to capture ‘events’ that lay outside the variance of the parent strain of Chlorella vulgaris, i.e. the control (no events in control). Three gates were captured (P4, P5, P6), with increasing distance from the parent strain of Chlorella vulgaris population (less fluorescence). Further, single cells from gates P5 and P6 were sorted into standard 24-well cell culture plates containing mixotrophic growth medium containing acetate as the carbon source, and incubated further. The incubation period was typically around 1 week. At the end of said incubation period, green-coloured cell cultures, indicating viable mutants (namely, p5-39, p5-59, p5-94 and p4-102), were obtained. Moreover, the cell cultures comprising viable mutants were subjected to a confocal microscopy analysis with calcofluor white staining, colorimetric assay or a combination thereof to quantify the overall chitin content in the mutants as compared to a chitin standard curve.

1. A modified strain of a Chlorella microalgae species, derived from a parent strain of a Chlorella microalgae species, the modified strain having a chitin content at least 10% lower than the chitin content of the parent strain of Chlorella microalgae from which it is derived, when grown under the same conditions.

2. A modified strain of a Chlorella microalgae species of claim 1, wherein the modified strain of the Chlorella microalgae is obtained from the parent strain by performing mutagenesis of the parent strain.

3. A modified strain of Chlorella microalgae species of claim 1 or 2, wherein the mutagenesis is performed by exposure of the parent strain of the Chlorella microalgae species to a sub-lethal quantity of a mutagenic chemical.

4. A modified strain of a Chlorella microalgae species of claim 3, wherein the mutagenic chemical is an alkylating agent.

5. A modified strain of a Chlorella microalgae species of claim 3 or 4, wherein the sub-lethal quantity of the mutagenic chemical is in a range of 0.1 to 2.0 M.

6. A modified strain of a Chlorella microalgae species of claim 2, wherein the mutagenesis is performed by exposure of the parent strain to a physical mutagen, wherein the physical mutagen comprises at least one of: UV light, gamma rays, X-rays.

7. A modified strain of a Chlorella microalgae species of claim 1, wherein the chitin content of the modified strain is a result of a stable genetic mutation.

8. A modified strain of a Chlorella microalgae species of any preceding claim, wherein the modified strain of Chlorella microalgae species has one or more additional desirable phenotypes, wherein the one or more additional desirable phenotypes is selected from a group comprising: a colour, a pigment content, a protein content, a smell, a taste, a texture, a biochemical composition and improved tolerance to process conditions.

9. A modified strain of a Chlorella microalgae species of any preceding claim, wherein the modified strain has a chlorophyll content in a range of 0.25 to 0.50 mg/g dry cell weight, 0.10 to 0.25 mg/g dry cell weight or 0.001 to 0.1 mg/g dry cell weight.

10. A modified strain of Chlorella microalgae species of any preceding claim, wherein the modified strain has a protein digestibility-corrected amino acid score (PDCAAS) in a range of 0.75 to 1.

11. A modified strain of Chlorella microalgae species of claim 10, wherein the modified strain the protein digestibility-corrected amino acid score is in a range of 0.75 to 0.831, preferably in a range of 0.75 to 0.79.

12. A modified strain of Chlorella microalgae species of any preceding claim, wherein the modified strain is cultivated in autotrophic, mixotrophic or a heterotrophic growth modes.

13. A modified strain of a Chlorella microalgae species of any preceding claims, wherein the modified strain is cultivated:
at a specific temperature,
for a predefined period of time,
optionally, without the presence of light, and
in the presence of an organic carbon energy source.

14. A modified strain of a Chlorella microalgae species of claim 13, wherein the specific temperature is in a range of 20 to 35° C., preferably in the range of 25 to 28° C., and most preferably above 28° C.

15. A modified strain of a Chlorella microalgae species of claim 13, wherein the predefined period of time is in a range of 1 to 5 weeks, preferably in the range of 1 to 3 weeks.

16. A modified strain of a Chlorella microalgae species of claim 13, wherein the organic carbon energy source is glucose or acetate.

17. A modified strain of a Chlorella microalgae species of any preceding claim, wherein the modified strain is genetically stable.

18. A modified strain of a Chlorella microalgae species of any preceding claim, wherein the modified strain has a stable genetic mutation affecting one or more of: a primary amino acid sequence of chitin synthase, a protein involved in the regulation of chitin synthase, in the biosynthesis of N-acetylglucosamine or its activated nucleotide sugar, in the metabolic flux of N-acetylglucosamine in the biosynthesis of chitin, or in the biosynthesis or regulation of a chitinase or related enzyme class.

19. A modified strain of a Chlorella microalgae species of any preceding claim, wherein the modified strain has a stable genetic mutation affecting an ABC transporter exhibiting a pleiotropic effect on chitin biosynthesis or turnover.

20. A modified strain of a Chlorella microalgae species of any preceding claim, wherein the modified strain has a stable genetic mutation affecting a protein subunit functioning as a guanine nucleotide-binding protein with a pleiotropic effect on chitin biosynthesis or turnover.

21. A modified strain of a Chlorella microalgae species of any preceding claim, wherein the modified strain has a stable genetic mutation affecting an aquaporin exhibiting a pleiotropic effect on chitin biosynthesis or turnover.

22. A modified strain of a Chlorella microalgae species of any preceding claim, wherein the Chlorella microalgae species is Chlorella vulgaris.

23. A modified strain of Chlorella vulgaris of claim 22 having a chitin content of less than 4.8 mg/g dry cell weight.

23. A modified strain of Chlorella vulgaris of claim 23, wherein the modified strain of Chlorella vulgaris has a chitin content in a range of 0.001 to 4.8 mg/g dry cell weight.

24. A modified strain of a Chlorella microalgae species of any of claims 1-21, wherein the Chlorella microalgae species is Chlorella sorokiniana.

25. A modified strain of Chlorella sorokiniana of claim 24 having a chitin content of less than 5.2 mg/g dry cell weight.

26. A modified strain of Chlorella sorokiniana of claim 25, wherein the modified strain of Chlorella sorokiniana has a chitin content in a range of 0.001 to 5.2 mg/g dry cell weight.

27. A modified strain of a Chlorella microalgae species of any of claims 1-21, wherein the microalgae is selected from a group comprising: Parachlorella kessleri, Auxenochlorella protothecoides, Auxenochlorella pyrenoidosa, or Heterochlorella luteoviridis.

28. A modified strain of microalgae of any preceding claim, wherein the modified strain is genetically stable and exhibits improved permeability to exogenous DNA, RNA, protein, polypeptides or complexes derived therefrom as compared to its parent strain.

29. A modified strain of microalgae of any preceding claim, wherein the modified strain is improved with regard to the energy required to process the resulting biomass for any application, as compared to its parent strain.

30. A method of producing a modified strain of a Chlorella microalgae species, the modified strain having a chitin content at least 10% lower than the chitin content of the parent strain of Chlorella microalgae from which it is derived, when grown under the same conditions, wherein the method comprises:
a) obtaining a parent strain of Chlorella microalgae;
b) performing mutagenesis of the parent strain;
c) cultivating the mutated strain: at a specific temperature, for a predefined period of time, and in the presence of an organic carbon source; and
d) identifying and isolating chitin-deficient mutants of the parent strain of the Chlorella microalgae.

31. A method according to claim 30, wherein the mutagenesis is performed by exposure of the parent strain of microalgae to a sub-lethal quantity of a mutagenic chemical.

32. A method of any of claim 30 or 31, wherein the mutagenic chemical is an alkylating agent.

33. A method of claim 30, wherein mutagenesis is performed by exposure of the parent strain to a physical mutagen, wherein the physical mutagen comprises at least one of: UV light, gamma rays, X-rays.

34. A method of any of claims 30 to 33, wherein the identification of the modified strain of the Chlorella microalgae comprises calcofluor white staining of the cells and sorting of the cells with flow cytometry.

35. A method of claim 34, wherein the method further comprises selecting healthy cells of the modified strain of the Chlorella microalgae.

36. A method of any of claims 30 to 35, wherein the method further comprises performing steps (b) to (d) of claim 30 repeatedly.

37. A method of any of claims 30 to 36, wherein the modified strain is cultivated using a heterotrophic growth medium.

38. A method of claim 30, wherein the specific temperature is in a range of 20 to 35° C., preferably in the range of 25 to 28° C., and most preferably above 28° C.

39. A method of claim 30, wherein the predefined period of time is in a range of 1 to 5 weeks, preferably in the range of 1 to 3 weeks.

40. A method of claim 30, wherein the organic carbon energy source is glucose or acetate.

41. A method according to any of claims 30 to 40 wherein the Chlorella microalgae species is Chlorella vulgaris and the modified strain has a chitin content of less than 4.8 mg/g dry cell weight.

42. A method according to any of claims 30 to 40 wherein the Chlorella microalgae species is Chlorella sorokiniana.

43. A method according to any of claims 30 to 40 wherein the Chlorella microalgae species is selected from a group comprising: Parachlorella kessleri, Auxenochlorella protothecoides, Auxenochlorella pyrenoidosa, or Heterochlorella luteoviridis.

44. A composition comprising an algae biomass derived from the modified strain of any one of claims 1 to 30 or obtained by performing the method of any of claims 31 to 43.

45. A composition of claim 44, wherein the composition is a food or food ingredient.

46. A composition of claim 44, wherein the composition is employed in at least one of: human foods, human nutraceutical preparations or formulations, animal feeds, pharmaceutical compositions including vaccines, cosmetics, personal care compositions, personal care devices.

47. A method of using the composition of claim 44 as an ingredient in at least one of: human foods, human nutraceutical preparations or formulations, animal feeds, pharmaceutical compositions including vaccines, cosmetics, personal care compositions, personal care devices or textiles, dyes, inks or fuels.

48. A microalgae flour comprising a homogenate of microalgae biomass derived from the modified strain of Chlorella vulgaris of any of claims 1 to 30 or obtained by performing the method of any of claims 31 to 43.

49. A modified strain of Chlorella vulgaris having a chitin content of less than 4.8 mg/g dry cell weight.
